(19) United States
(12) Patent Application Publication
(10) Pub. No.: US 2023/0381201 A1
(43) Pub. Date: Nov. 30, 2023

(54) METHODS AND COMPOSITIONS FOR TREATING FIBROTIC DISEASES

(71) Applicant: MAX BioPharma, Inc., Los Angeles, CA (US)

(72) Inventors: Farhad Parhami, Los Angeles, CA (US); Frank Stappenbeck, Los Angeles, CA (US); Feng Wang, Los Angeles, CA (US)

(73) Assignee: MAX BioPharma, Inc., Los Angeles, CA (US)

(21) Appl. No.: 18/033,885

(22) PCT Filed: Nov. 4, 2021
(86) PCT No.: PCT/US2021/058106

§ 371 Date: Apr. 26, 2023

Related U.S. Application Data
(60) Provisional application No. 63/110,147, filed on Nov. 5, 2020.

(51) Int. Cl. A61K 31/58; A61K 45/06; A61P 11/00

(52) U.S. Cl. CPC A61K 31/58; A61K 45/06; A61P 11/00

(57) ABSTRACT

Described herein are methods and compositions for treating fibrotic diseases and disorders.

### TECHNICAL FIELD

The present disclosure relates to methods and compositions for treating fibrotic diseases and disorders.

### BACKGROUND

Increasingly significant disease in the U.S. population with an estimated 30-40% of adults affected by Non-Alcoholic Fatty Liver Disease, 6% of which have NASH. It is a silent disease with no real symptoms and is characterized by steatosis, inflammation and ballooning of hepatocyte cells. NASH is expected to increase by 63% between 2015 and 2030 in the U.S. (Estes C et al. Hepatology 67:123-133; 2018). Currently, there are no safe drugs that can reverse liver fibrosis, once scarring has reached a certain point, liver transplant becomes the only treatment option. Direct medical cost of liver fibrosis in U.S. adults is $103B annually (Younossi Z M et al. Hepatology 64:1577-1586; 2016) and another $188B in indirect costs related to liver fibrosis (Shetty A and Syn W. Federal Practitioner 36:14-19; 2019).

Tissue fibrosis that occurs as a result of uncontrolled wound healing is an unmet medical need that can occur in multiple organs and tissues including liver, lung, kidney, and skin as a result of acute or chronic injury. Liver fibrosis including non-alcoholic liver steatohepatitis (NASH) is a major health problem that causes morbidity and mortality. Two key mediators of liver fibrosis have been identified as activation of Hedgehog (Hh) and TGFb signaling. Inhibition of these pathways using specific antagonists have shown amelioration of fibrosis process. A number of cells in the liver have been recognized as responsible for fibrosis. Noteworthy are myofibroblasts and stellate cells that upon activation by TGFb and Hh ligands express a number of fibrogenic molecules including connective tissue growth factor (CTGF), which is considered a drug target for intervention in fibrosis.

An estimated 37 million American adults (15% of adult population) have Chronic Kidney Disease (CKD) and its prevalence is more than 50% among adults above the age of 70 years. Progressive kidney disease frequently results in renal fibrosis and, eventually, renal failure. A healthy kidney has healthy tubules, preserved capillary density, inimal interstitial extracellular matrix, minimal matrix cross-linking, soft extracellular matrix and minimal oxygen diffusion distance to tubular cells. In contrast, a fibrotic kidney has dilated, atrophic tubules, reduced capillary density, increased interstitial extracellular matrix, increased matrix cross-linking, stiff extracellular matrix and increased oxygen diffusion distance to tubular cells.

In 2017, combined Medicare costs of CKD were $84 billion. Each year, kidney disease kills more people than breast or prostate cancer. For example, in 2013, more than 47,000 Americans died of kidney disease. Kidney disease has few symptoms in its early stages and is often left untreated until more advanced, eventually requiring dialysis or a kidney transplant. The global CKD drugs market was valued at $12.4 billion USD in 2016 and is expected to reach $17.4 billion USD by 2025. However, currently available drugs only ameliorate or delay the progression of CKD but do not stop or reverse fibrosis.

Idiopathic pulmonary fibrosis (IPF) is a complex chronic fibroproliferative lung disease of unknown etiology, with an increasing incidence. The disease is characterized by progressive accumulation of extracellular matrix within the interstitium. It is a progressive and irreversible disease with an estimated median survival of only 36 months. Historically, corticosteroids in combination with immunosuppressives and/or N-acetylcysteine, have been advocated as reasonable, but unproven treatment strategy for IPF. It is clear that better and more effective treatments for IPF are needed.

Pancreatic fibrosis, a characteristic histopathological feature of chronic pancreatitis, is actually an active dynamic process that results in irreversible morphological scarring of the pancreatic parenchyma. The long-standing or recurrent inflammation of the pancreas is often associated with progressive fibrosis; thus, causes persistent abdominal pain and permanent impairment of pancreatic functions, and eventually leads to a variety of systemic complications including malabsorption and diabetes mellitus. In general, chronic pancreatitis is commonly arisen from abnormal or over-consumption of alcohol. Besides inflammation, pancreatic fibrosis can also be idiopathic and hereditary. It is known in the art that chronic pancreatitis and fibrosis are linked with an increased risk of pancreatic cancer and effective treatments for pancreatic fibrosis are needed.

Thus, there is clearly a need in the art for new, effective methods and compositions for treating or preventing fibrotic disorders. The present disclosure addresses some of these needs.

### SUMMARY

In one aspect provided herein is a method for treating a fibrotic disease or disorder in a subject. Generally, the method comprises administering a therapeutically effective amount of a compound of Formula (I), or a pharmaceutically acceptable salt or solvate thereof, to a subject in need thereof. The compound of Formula (I) is of chemical structure:

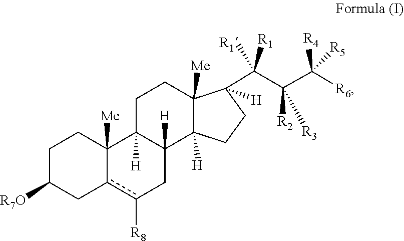

wherein:
[symbol] is a single or double bond; R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, provided that one of R₁ and R₁′ is OH;
R₂, R₃, R₄, and R₅ are independently hydrogen, deuterium, C₁-C₈alkyl, or —OH, or one of R₂ or R₃ together with one of R₄ or R₅ forms a double bond;
R₆ is alkyl, aryl or heteroaryl, wherein the alkyl, aryl or the heteroaryl are optionally substituted with 1, 2, 3, or 4 R₉ groups;
R₇ is hydrogen, substituted or unsubstituted C₁-C₈alkyl, or —C(O)NR₁₀R₁₁;
R₈ is hydrogen or —OH;
each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄);
R₁₀ and R₁₁ are independently hydrogen, substituted or unsubstituted C₁-C₈alkyl, or substituted or unsubstituted aryl;
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects, the compound of Formula I is Oxy210 having the chemical structure:

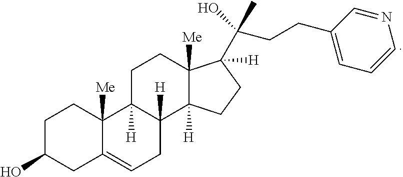

The methods and compositions described herein can be used for treating any number of fibrotic diseases or disorders. For example, the methods and compositions described herein can be used for treating liver, lung, kidney, pancreas, or skin fibrosis. The methods and compositions described herein can be used for treating fibrosis that is a result of acute or chronic injury. For example, lung fibrosis associated with chemotherapy or radiotherapy or antibiotic therapy, liver fibrosis associated with chronic viral infection, pancreas fibrosis associated with pancreatic cancer, and lung fibrosis associated with lung viral infection, e.g., coronavirus infection.

In some embodiments, the methods and compositions described herein can be used for treating bleomycin-induced pulmonary fibrosis.

It is noted that the compound of Formula (I) can be adminstered to a subject having or in need of treatment for a fibrotic disease. The compound of Formula (I) can also be administered as a prophylaxis to a subject having increased risk of developing fibrosis such as subjects with metabolic diseases, diabetes and obesity.

The compounds descried herein inhibit the expression of collagen, fibronectin, laminin, connective tissue growth factor (CTGF) and other extracellular matrix proteins associated with tissue fibrosis. Thus, the compounds can be used in methods to inhibit expression and/or level of one or more of these. For example, a compound of Formula (I), or a pharmaceutically acceptable salt or solvate thereof, can be administered to a cell for inhibiting the expression and/or level of collagen, fibronectin, laminin, connective tissue growth factor (CTGF) and/or other extracellular matrix proteins associated with tissue fibrosis. It is noted that administering to the cell can be in vitro or in vivo. For example, when the administering to the cell is in vivo, the compound can be administered to a subject.

### BRIEF DESCRIPTION OF THE DRAWINGS

FIG. 1 is a bar graph showing inhibition of basal expression of TGFβ target genes by an exemplary orally bioavailable oxysterol, Oxy210, in primary human liver stellate cells. THBS1: Thrombospondin 1; LOX: Lysyl Oxidase; COL1A1: Collagen 1A1; and CTGF: Connective Tissue Growth Factor. Data from a representative of 3 separate experiments are reported as the mean of triplicate determinations ±SD (**p<0.01 VS. control).

FIG. 2 is a bar graph showing inhibition of basal expression of profibrogenic genes by Oxy210 in primary human pericytes. Data from a representative experiment are reported as the mean of triplicate determinations ±SD (**p<0.01 vs. control).

FIG. 3 is a bar graph showing effect of Oxy210 on TGFβ-induced expression of profibrotic genes in primary human pericytes. Data from a representative experiment are reported as the mean of triplicate determinations ±SD (##p<0.01 vs. control; **p<0.01 VS. TGFβ).

FIG. 4 is a bar graph showing inhibition of TGFβ-induced epithelial mesenchymal transition (EMT) of renal tubular epithelial cells by Oxy210. Data from a representative experiment are reported as the mean of triplicate determinations ±SD (##p<0.01 VS. control; ** p<0.01 vs. TGFβ).

FIG. 5 is a line graph showing inhibition of IMR-90 normal human lung fibroblast cell proliferation by Oxy210. Data from a representative experiment are reported as the mean of triplicate determinations ±SD (**p<0.01 vs. control).

FIG. 6 is a line graph showing inhibition of proliferation of LL97A human lung fibroblast cells derived from a subject with idiopathic pulmonary fibrosis by Oxy210. Data from a representative experiment are reported as the mean of triplicate determinations ±SD (*p<0.05 vs. control; **p<0.01 vs. control).

FIG. 7 is a bar graph showing Oxy210 inhibits basal profibrotic gene expression in IMR-90 cells. Data from a representative experiment are reported as the mean of triplicate determinations ±SD (*p<0.05 vs. control; **p<0.01 vs control).

FIG. 8 is a bar graph showing Oxy210 inhibits TGFβ-induced COL1A1 and a-SMA expression in LL97A cells. Data from a representative experiment are reported as the mean of triplicate determinations ±SD (*p<0.05 vs. TGFβ treated cells; **p<0.01 vs. TGFβ treated cells).

FIG. 9 is a line graph showing Oxy210 inhibits basal COL1A1 expression in LL97A cells. Data from a representative experiment are reported as the mean of triplicate determinations ±SD (**p<0.01 vs control).

FIG. 10 are photographs showing the effect of Oxy210 on bleomycin-induced lung pathology in mice.

FIGS. 11A-11D show effect of Oxy210 on bleomycin-induced pulmonary fibrosis in mice. Body weights were measured during and at the end of the study (FIGS. 11A and 11B), Ashcroft scoring performed at the end of the study (FIG. 11C), and macrophage numbers in the bronchoalveolar lavage fluid (BALF) determined at the end of study (FIG. 11D).

### DETAILED DESCRIPTION

The compounds of Formula (I) can be used in methods and compositions for treatment of a fibrotic disease or disorder. As used herein, the term “fibrotic disease” or “fibrotic disorder” refers to a medical condition featuring progressive and/or irreversible fibrosis, wherein excessive deposition of extracellular matrix occurs in and around inflamed or damaged tissue. In certain embodiments, a fibrotic disorder or disease is associated with the persistent presence of myofibroblasts in and around fibrotic foci or lesions. Excessive and persistent fibrosis can progressively remodel and destroy normal tissue, which may lead to dysfunction and failure of affected organs, and ultimately death. A fibrotic disorder may affect any tissue in the body and is generally initiated by an injury and the transdifferentiation of fibroblasts into myofibroblasts. As used herein, “transdifferentiation” refers to the direct conversion of one cell type into another. It is to be understood that fibrosis alone triggered by normal wound healing processes that has not progressed to a pathogenic state is not considered a fibrotic disorder or disease of this disclosure. A “fibrotic lesion” or “fibrotic plaque” refers to a focal area of fibrosis.

It is noted the compound can be administered to the subject after a fibrotic lesion has developed in the subject. Alternatively, or in addition, the compound can be administered prophylactically to the subject patients with increased risk of developing fibrosis such as subjects with metabolic diseases and obesity

Exemplary fibrotic diseases or disorders include, but are not limited to, acute interstitial pneumonitis, adhesive capsulitis, asthma, arthrofibrosis (e.g., knee, shoulder, other joints), atrial fibrosis, bone-marrow fibrosis, cardiac fibrosis, chronic kidney disease, cirrhosis of liver and gallbladder, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, hypertrophic scarring, idiopathic interstitial fibrosis, idiopathic pulmonary fibrosis (IPF), interstitial lung disease, interstitial pneumonitis, lung fibrosis associated with chemotherapy or radiotherapy or antibiotic therapy, lung fibrosis associated with lung viral infection (for example coronavirus infection), ischemia associated fibrosis, keloid, liver fibrosis (e.g., cirrhosis), non-alcoholic steatohepatitis (NASH), alcoholic steatohepatitis, liver fibrosis associated with chronic viral infection, lymphocytic interstitial pneumonia, mediastinal fibrosis, muscle fibrosis, myelofibrosis, nephrogenic systemic fibrosis, nonspecific interstitial pneumonia, organ transplant associated fibrosis, organ transplant fibrosis, pancreatic fibrosis or pancreatic ductal adenocarcinoma (PDA), Peyronie's disease, progressive massive fibrosis (e.g., lungs), pulmonary fibrosis, renal fibrosis, respiratory bronchiolitis, retroperitoneal fibrosis, retroperitoneal fibrosis, scleroderma, systemic sclerosis (e.g., skin, lungs), pancreatic cancer, and vascular fibrosis (e.g., atherosclerosis, stenosis, restenosis).

In some embodiments of any one of the aspects, the fibrotic disease or disorder is liver, lung, kidney, pancreas, or skin fibrosis.

In some embodiments, the fibrotic disease or disorder is pulmonary fibrosis, liver fibrosis, including non-alcoholic steatohepatitis (NASH) and alcoholic steatohepatitis, idiopathic pulmonary fibrosis (IPF), pancreatic fibrosis, cardiac fibrosis, or kidney fibrosis.

It is noted that the fibrosis can be anywhere in the subject. For example, the fibrosis can be at site of an injury to a tissue. For example, the fibrosis can be at site of an injury to a tissue selected from the group consisting of: liver tissue, upper respiratory system tissue, lower respiratory system tissue, lung tissue, central nervous system tissue, eye tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue. In some embodiments, the injured tissue is lung tissue, liver tissue, or kidney tissue.

In some embodiments, fibrotic disease is bleomycin-induced pulmonary fibrosis.

### Combination Therapy

In some embodiments of any one of the aspects, a method described herein further comprises administering, e.g., co-adminstering, at least one additional therapeutic to the subject.

In some embodiments of any one of the aspects, the additional therapeutic is an anti-fibrotic agent. Exemplary additional anti-fibrotic agents include, without limitation, calcium channel blockers, cytotoxic agents, cytokines, chemokines, integrins, growth factors, hormones, lysophosphatidic acid (“LPA”) receptor 1 antagonists, agents that modulate the TGF-β pathway, endothelin receptor antagonists, agents that reduce connective tissue growth factor (“CTGF”) activity, matrix metalloproteinase (“MMP”) inhibitors, agents that reduce the activity of platelet-derived growth factor (“PDGF”), agents that interfere with integrin function, agents that interfere with the pro-fibrotic activities of cytokines, agents that reduce oxidative stress, PDE4 inhibitors, PDE5 inhibitors, mTor inhibitors, modifiers of the arachidonic acid pathway, peroxisome proliferator-activated receptor (“PPAR”)-γ agonists, kinase inhibitors, inhibitors of VEGF signaling pathway, matrix metalloproteinases, tissue inhibitors of metalloproteinases (“TIMPs”), HGF agonists, angiotensin-converting enzyme (“ACE”) inhibitors, angiotensin receptor antagonists, inhibitors of advanced glycation endproducts (“AGEs”) or their receptors (“RAGEs”), Rho kinase inhibitors, PKC inhibitors, ADAM-10 inhibitor, farnesoid X receptor agonists, caspase inhibitors, anti-oxidants, inhibitors of collagen expression, LMW heparin or heparin analogs, copper chelators, TNF-α blocking agents, agents that inhibit fibronectin deposition and/or enhance fibronectin degradation and turnover (e.g., bacterial adhesin peptides, fibronectin-derived peptides, and antibodies against fibronectin, as described in U.S. Appl. Publ. No. 20130190224 to Sottile et al., which is hereby incorporated by reference in its entirety), HMG-CoA reductase inhibitors, Thy-1 (CD90) inhibitors, and antiviral agents.

Exemplary calcium channel blockers include, without limitation, Verapamil.

Exemplary cytotoxic agents include, without limitation, azathioprine, methotrexate, and cyclophosphamide.

Exemplary cytokines include, without limitation, interleukins such as IL-1, IL-4, IL-5, IL-6, IL-8, IL-10, IL-12, and IL-13; interferons such as interferon-γ; lymphokines; tumor necrosis factor-α; endothelin-1; angiotensin II; leptins; angiogenin(s); monocyte chemoattractant protein type 1 (MCP-1); and macrophage inflammatory protein (MIP-1α, MIP-2).

Exemplary chemokines include, without limitation, CCL2, CCL12, CXCL12, CXCR4, CCR3, CCR5, CCR7, and SLC/CCL21.

Exemplary integrins include, without limitation, α₁β₁, α₂B₁, αᵥβ₆, and αᵥβ₃. Exemplary growth factors include, without limitation, insulin growth factors (IGF-1, IGF-2), keratinocyte growth factor (“KGF”), hepatocyte growth factor (“HGF”), fibroblast growth factors (FGF-1, 2 and 4), platelet-derived growth factors (PDGF-AB, PDGF-BB, PDGF-AA), epidermal growth factors (“EGFs”), transforming growth factors (TGF-β1-3), osteoid-inducing factor (“OIF”), bone morphogenic proteins (BMPs; BMP1, BMP2, BMP2A, BMP2B, BMP3, BMP3b, BMP4, BMP5, BMP6, BMP9-BMP-15, OP-1, OP-2, OP-3, BMP-7, HBGF-1, HBGF-2), growth differentiation factors (GDF1-3 and GDF5-12), osteogenic proteins (OP-1, OP-2, OP-3), cartilage-derived morphogenic proteins (CDMP-1, CDMP-2, CDMP-3), colony stimulating factors (CSF-1, G-CSF and GM-CSF or isoforms thereof), vascular endothelial growth factor (“VEGF”), connective tissue growth factor (“CTGF”), and neural epidermal growth factor-like 1 (NELL-1).

Exemplary hormones include, without limitation, progesterone, estrogen, testosterone, growth hormone, thyroid hormone, and parathyroid hormone.

Exemplary lysophosphatidic acid (LPA) receptor 1 antagonists include, without limitation, AM152 (Amira Pharmaceuticals), AM966, and Ki16198.

Exemplary agents that modulate TGF-β pathways include, without limitation, αᵥβ₆inhibitors; HGF; rBMP7 (bone morphogenic protein 7); decorin; tyrosine kinase inhibitors (Imantinib, Desatinib, Nolitinib); and agents that that reduce TGF-β activity (e.g., metelimumab (CAT-192), GC-1008 (Genzyme/Medimmune), lerdelimumab (CAT-152), LY-2157299 (Eli Lilly), and ACU-HTR-028 (Opko Health)); antibodies that target one or more TGF-β isoforms; inhibitors of TGF-β receptor kinases (e.g., TGFBR1 (ALK5) and TGFBR2); modulators of post-receptor signaling pathways; and chemokine receptor signaling.

Exemplary endothelin receptor antagonists (including inhibitors that target both endothelin receptor A and B and those that selectively target endothelin receptor A) include, without limitation, ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; and zibotentan.

Exemplary agents that reduce the activity of connective tissue growth factor (CTGF) include, without limitation, FG-3019, FibroGen, other CTGF-neutralizing antibodies.

Exemplary matrix metalloproteinase (MMP) inhibitors include, without limitation, MMPI-12, PUP-1 and tigapotide triflutate.

Exemplary agents that reduce the activity of platelet derived growth factor (“PDGF”) include, without limitation, Imatinib mesylate (Novartis)) and PDGF neutralizing antibodies, antibodies targeting PDGF receptor (“PDGFR”), inhibitors of PDGFR kinase activity, and post-receptor signaling pathways. PDGFR inhibitors include, but are not limited to, SU9518, CP-673,451 and CP-868596.

Exemplary agents that interfere with integrin function include, without limitation, STX-100, IMGN-388, and integrin targeted antibodies.

Exemplary agents that interfere with the pro-fibrotic activities of cytokines (such as interleukins, e.g., IL4 and IL-13) include, without limitation, AER-001, AMG-317, APG-201, sIL-4Rα, anrukinzumab, CAT-354, cintredekin besudotox, MK-6105, QAX-576, SB-313, SL-102, and TNX-650; as well as neutralizing antibodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway.

Exemplary agents that interfere with epithelial mesenchymal transition include, without limitation, inhibitors of mTor (including but not limited to rapamycin, 40-O-(2-hydroxy)-ethyl-rapamycin, 32-deoxorapamycin, 40-[3-hydroxy-2-(hydroxy-methyl)-2-methylpropanoate]-rapamycin, Ridaforolimus (AP-23573 or MK-8669) and Torisel (temsirolimus).

Exemplary agents that reduce oxidative stress include, without limitation, N-acetylcysteine (a cysteine pro-drug), tetrathiomolybdate, and interferon-γ.

Exemplary agents that are inhibitors of phosphodiesterase 4 (PDE4) or phosphodiesterase 5 (“PDE5”) include, without limitation, Roflumilast, mirodenafil, PF-4480682, sildenafil citrate, SLx-2101, tadalafil, udenafil, UK-369003, vardenafil, and zaprinast.

Exemplary modifiers of the arachidonic acid pathway include, with limitation, cyclooxygenase (“COX”) and 5-lipoxygenase (“LO”) inhibitors such as Zileuton.

Exemplary compounds that reduce tissue remodeling during fibrosis include, without limitation, prolyl hydrolase inhibitors such as 1016548, CG-0089, FG-2216, FG-4497, FG-5615, FG-6513, fibrostatin A (Takeda), lufironil, P-1894B, and safironil.

Exemplary PPAR-gamma agonists include, without limitation, pioglitazone (Takeda), farglitizar (GSK) and rosiglitazone (GSK).

Exemplary kinase inhibitors include, without limitation, MEK inhibitors (e.g., PD325901, ARRY-142886, ARRY-438162 and PD98059); EGFR inhibitors (e.g., Iressa™ (gefitinib, AstraZeneca), Tarceva™ (erlotinib or OSI-774, OSI Pharmaceuticals Inc.), Erbitux™ (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFR immunoliposomes (Hermes Biosciences Inc.) and combinations thereof, antibodies targeting EGF receptor, inhibitors of EGF receptor kinase, and modulators of post-receptor signaling pathways); ErbB2 receptor inhibitors (e.g., CP-724-714, CI-1033 (canertinib), Herceptin™ (trastuzumab), Omnitarg™ (2C4, petuzumab), TAK-165, GW-572016 (Ionafarnib), GW-282974, EKB-569, PI-166, dHER2 (HER2 Vaccine), APC8024 (HER2 Vaccine), anti-HER/2neu bispecific antibody, B7.her2IgG3, AS HER2 trifunctional bispecific antibodies, mAB AR-209 and mAB 2α-1); IGFIR antibodies (e.g., those described in PCT Application Publ. No. WO 2002/053596, which is hereby incorporated by reference in its entirety); AXL inhibitors (e.g., SGI-AXL-277 (SuperGen) as well as inhibitors disclosed in U.S. Pat. Pub. 20050186571, which is hereby incorporated by reference in its entirety); p38 inhibitors (e.g., SB202190, SB203580 and pyridinyl imidazoles); FGFR inhibitors (e.g., PD 17034, PD166866, and SU5402); TIE2 inhibitors (e.g., those described in Kissau, L. et. al., J Med Chem., 46:2917-2931 (2003), which is hereby incorporated by reference in its entirety); the following kinase inhibitors: Pan ERBB receptor inhibitors (e.g., GW572016, CI-1033, EKB-569, and Omnitarg), MP371 (SuperGen) which is an inhibitor of c-Kit, Ret, PDGFR, and Lck, as well as the non-receptor tyrosine kinase c-src, MP470 (SuperGen) which is an inhibitor of c-Kit, PDGFR, and c-Met, Imatinib (Gleevec™) which is an inhibitor of c-kit, PDGFR, and ROR, as well as the non-receptor tyrosine kinase bcl/abl, Lapatinib (Tykerb™) which is an epidermal growth factor receptor (EGFR) and ERBB2 (Her2/neu) dual tyrosine kinase inhibitor, inhibitors of PDGFR and VEGFR (e.g., Nexavar™ (sorafenib, BAY43-9006), Sutent™ (sunitinib, SU11248), and ABT-869), inhibitors of VEGFR and (e.g., Zactima™ (vandetanib, ZD-6474), BMS-690514 which is a reversible oral inhibitor of epidermal growth factor receptor (“EGFR/HIER-1”), HER-2 and -4, and vascular endothelial growth factor receptors (“VEGFRs”)-1 to -3, BIBF-1120 which is a receptor kinase inhibitor for VEGF, FGF and PDGF; inhibitors of the VEGF signaling pathway (e.g., PTC-299, INGN-241, oral tetrathiomolybdate, 2-methoxyestradiol, 2-methoxyestradiol nanocrystal dispersion, bevasiranib sodium, PTC-299, Veglin, VEGF neutralizing antibodies, soluble form of VEGFR1 (sFlt) and derivatives thereof which neutralize VEGF, anti-KDR antibodies, VEGFR1 (F1t1) antibodies (e.g., icrucumab (IMC-18F1)), VEGFR2 (KDR) antibodies (e.g., CDP-791 or IMC-1121B (ramucirumab) and VEGFR3 antibodies (e.g., mF4-31C1 from Imclone Systems) and CT-322 (Angiocept™; a VEGFR2 inhibitor), VEGF inhibitors (e.g., bevacizumab (Avastin™) pegaptanib, ranibizumab, NEOVASTAT™, AE-941, VEGF Trap, and PI-88), and VEGF receptor antagonists (e.g., JNJ-17029259 (4-[4-(1-Amino-1-methylethyl)phenyl]-2-[4-(2-morpholin-4-yl-ethyl)phenylamino]pyrimidine-5-carbonitrile (a VEGF-R2 inhibitor)), PTK-787/ZK222584 (Astra-Zeneca), SU5416, SU11248 (Pfizer), ZD6474 ([N-(4-bromo-2-fluorophenyl)-6-methoxy-7-[(1-methylpiperidin-4-yl)methoxy]quinazolin-4-amine]), vandetanib, cediranib, AG-013958, CP-547632, E-7080 (lenvatinib), XL-184, L-21649, ZK-304709, SU6668, sorafenib, sunitinib, pazopanib, vatalanib, AEE-788, AMG-706 (motesanib), axitinib, BIBF-1120, SU-14813, XL-647, XL-999, ABT-869, BAY-57-9352, BAY-73-4506 (regorafinib), BMS-582664, CEP-7055, CHIR-265, OSI-930, TKI-258, fenretinide, and squalamine).

Suitable matrix degrading enzymes include those described in U.S. Application Publ. Nos. 20100003237 and 20120101325, each of which is hereby incorporated by reference in its entirety. Exemplary matrix degrading enzymes include, without limitation, pancreatic elastase, elastase-2a, elastase-2b, neutrophil elastase, proteinase-3, endogenous vascular elastase, cathepsin G, mast cell chymase, mast cell tryptase, plasmin, thrombin, granzyme B, cathepsin S, cathepsin K, cathepsin L, cathepsin B, cathespin C, cathepsin H, cathespin F, cathepsin G, cathepsin 0, cathepsin R, cathepsin V (cathepsin 12), cathepsin W, calpain 1, calpain 2, legumain, cathepsin Z (cathepsin X), cathepsin D, cathepsin E, chondroitinase ABC, chondroitinase AC, hyaluronidase, chymopapain, chymotrypsin, collagenase, papain, subtilisin, subtilisin A, heparanase. and matrix metalloproteinases, such as for example, MMP-1 (collagenase-1), MMP-2 (gelatinase A), MMP-3 (stromelysin-1), MMP-7 (matrilysin; PUMP1), MMP-8 (collagenase-2), MMP-9 (gelatinase B), MMP-10 (stromelysin-2), MMP-11 (stromelysin-3), MMP-12 (metalloelastase), MMP-13 (collagenase-3), MMP-14 (MT1-MMP), MMP-15 (MT2-MMP), MMP-16 (MT3-MMP), MMP-17 (MT4-MMP), MMP-18 (collagenase-4), MMP-19 (stromelysin-4), MMP-20 (enamelysin), MMP-21 (x-MMP), MMP-23A (MT5-MMP), MMP-23B, MMP-24 (MT5-MMP), MMP-25 (MT6-MMP), MMP-26 (matrilysin-2), MMP-27 (MMP-22; c-MMP), MMP-28 (epilysin), ADAMTS-1, ADAMTS-2, ADAMTS-3, ADAMTS-4 (aggrecanase-1), ADAMTS-5 (aggrecanase-2), ADAMTS-14.

Exemplary tissue inhibitors of matrix-metalloproteinases (TIMPs) include, without limitation, TIMP-1, TIMP-2, TIMP-3, and TIMP-4.

Exemplary HGF agonists include, without limitation, Refanalin (Angion Biomedica).

Exemplary ACE inhibitors include, without limitation, Alacepril, Benazepril, Captopril, Cilazapril, Ceronapril, Delapril, Enalapril, Enalaprilat, Fosinopril, Fosinoprilat, Imidapril, Lisinopril, Moexipril, Perindopril, Perindoprilat, Quinapril, Quinaprilat, Ramipril, saralasin acetate, spirapril, temocapril, trandolapril, fasidotrilat, beclometasone dipropionate, FPL-66564, Idrapril, MDL-100240, and S-5590.

Exemplary angiotensin receptor antagonists include, without limitation, Candesartan, Irbesartan, Losartan, Valsartan, Telmisartan, and Eprosartan.

Exemplary advanced glycation endproducts (AGEs) inhibitors include, without limitation, Pyridoxamine (Biostratum). Examples of AGE receptors (RAGE) inhibitors include, without limitation, TTP-488 (Transtech Pharma) and TTP-4000 (Transtech Pharma).

Exemplary Rho kinase inhibitors include, without limitation, GSK269962, GSK429286, AS 1892802, SB772077B, and SR3677.

Exemplary PKC inhibitors include, without limitation, Ruboxistaurin mesilate hydrate (Lilly).

Exemplary ADAM-10 inhibitors include, without limitation, XL-784 (Exelixis).

Exemplary farnesoid X receptor agonists include, without limitation, INT-747 (Intercept Pharmaceuticals).

Exemplary caspase inhibitors include, without limitation, PF-3491390 (Pfizer, formally IDN-6556), and LB84318 (LG Life Sciences).

Exemplary anti-oxidants include, without limitation, Heptax (Hawaii Biotech), N-acetylcysteine (Pierre Fabre), tocopherol, silymarin, and Sho-saiko-To (H-09).

Exemplary inhibitors of collagen expression include, without limitation, Pirfenidone (InterMune), Halofuginone (Collgard) and F351 (Shanghai Genomics).

Exemplary low molecular weight heparin or heparin analogs include, without limitation, Sulodexide (Keryx).

Exemplary copper chelators include, without limitation, Trientine (Protemix), Coprexa (Pipex), and tetrathiomolybdate.

Exemplary TNF-α blocking agents include, without limitation, Etanercept (Enbrel™) and pentoxyfylline (Trental™).

Exemplary HMG-CoA reductase inhibitors include, without limitation, statins such atorvastatin (Lipitor), fluvastatin (Lescol), lovastatin (Mevacor, Altocor), pitavastatin (Livalo), pravastatin (Pravachol), rosuvastatin (Crestor), and simvastatin (Zocor).

Exemplary Thy-1 (CD90) inhibitors include monoclonal antibodies against Thy-1 (e.g., clone 5E10, Gundlach et al., Bioconjug. Chem. 22(8):1706-14 (2011), which is hereby incorporated by reference in its entirety).

Other known anti-fibrotic agents include, without limitation, 5-flurouracil (5-FU; a pyrimidine analog), mitomycin C (MMC), colchicine (antibiotic), d-penicillamine, Pediapred oral liquid, Medrol, cyclosporine (an immunosuppressant), mycophenolate mofetil (MMF; Cellcept; an immunosuppressant); prednisolone; bovine collagen type I, ribavirin (a guanosine (ribonucleic) analog), spirichostatin A (a histone deacetylase inhibitor); TGF-2 specific inhibitors (transglutaminase-2), tacrolimus (FK5-6, a calcineurin inhibitor), relaxin, taurine, niacin, treprostinil (a prostacyclin analog), Tiplaxtinin (PAI-039, a plasminogen activator-1 inhibitor); Pentraxin-1 (e.g., serum amyloid P component (SAP), c-reactive protein (CRP), and PTX-3) and Pentraxin-2 (PTX-2 or PRM-151), imidazolium and imidazolinium salts (U.S. Publication Application No. 20116178040, which is hereby incorporated by reference in its entirety) and IL-17 antagonists (U.S. Publication Application No. 20110091378, which is hereby incorporated by reference in its entirety); relaxin (a hormone; U.S. Publication Application No. 20120101325, which is hereby incorporated by reference in its entirety), ultraviolet A (UVA), pirfenidone, nintedabin, and cannabinoids and agents altering the MMP-TIMP balance.

The compounds of Formula (I) can inhibit the expression of genes and proteins associated with tissue fibrosis. Accordingly, a compound of Formula (I), or a pharmaceutically acceptable salt or solvate thereof, can be administered to a cell for inhibiting the expression of genes associated with tissue fibrosis. For example, a compound of Formula (I), or a pharmaceutically acceptable salt or solvate thereof can be administered to a cell for inhibiting the expression of one or more of collagen, fibronectin, laminin, connective tissue growth factor (CTGF) and/or other extracellular matrix proteins associated with tissue fibrosis.

It is noted that administering to the cell can be in vitro or in-vivo. Methods for administering a compound to a cell are well known and available to one of skill in the art. As used herein, administering the compound to the cell means contacting the cell with the compound so that the compound is taken up by the cell or the compound triggers responses by the cells with or without internalization. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises a compound of Formula (I). Where the cell is in vivo, “contacting” or “contact” includes administering the compound, e.g., in a pharmaceutical composition to a subject via an appropriate administration route such that the compound contacts the cell in vivo.

For example, when the cell is in vitro, said administering to the cell can include subjecting the cell to an appropriate culture media which comprises the indicated compound. Where the cell is in vivo, said administering to the cell includes administering the compound to a subject via an appropriate administration route such that the compound is administered to the cell in vivo. The cell to be administered a compound of Formula (I) can be any desired cell. In some embodiments of any one of the aspects, the cell is a liver cell. In some embodiments of any one of the aspects, the cell is a cell of the respiratory system. For example, the compound of Formula (I) is administered to ciliated cells, basal cells, epithelial cells, goblet cells and/or an alveolar cell. In some embodiments of any one of the aspects, the cell is a kidney cell. Cell can be selected from stellate cells and myofibroblasts myofibroblasts in liver, kidney, lung, or pancreas. It is noted that pericytes and pericyte-like cells that are present in almost all tissues can undergo fibrosis. Thus, the cell can be selected from pericytes and pericyte-like cells.

### Comounds

In compounds of Formula (I), [symbol] is a single or double bond; R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, provided that one of R₁ and R₁′ is OH; R₂, R₃, R₄, and R₅ are independently hydrogen, deuterium, C₁-C₈alkyl, or —OH, or one of R₂ or R₃ together with one of R₄ or R₅ forms a double bond; R₆ is alkyl, aryl or heteroaryl, wherein the alkyl, aryl or the heteroaryl are optionally substituted with 1, 2, 3, or 4 R₉ groups; R₇ is hydrogen, substituted or unsubstituted C₁-C₈alkyl, or —C(O)NR₁₀R₁₁; R₈ is hydrogen or —OH; each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄); R₁₀ and R₁₁ are independently hydrogen, substituted or unsubstituted C₁-C₈alkyl, or substituted or unsubstituted aryl; each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of the any one of the aspects described herein, [symbol] is a single. In some other embodiments of any one of the aspects described herein, [symbol] is a double bond.

In some embodiments of any one of the aspects described herein, R₁ is substituted or unsubstituted C₁-C₈alkyl. In some embodiments of any one of the aspects described herein, R₁ is substituted C₁-C₈alkyl. In some embodiments of any one of the aspects described herein, R₁ is —CF₃. In some embodiments of any one of the aspects described herein, R₁ is unsubstituted C₁-C₈alkyl. In some embodiments of any one of the aspects described herein, R₁ is unsubstituted C₁-C₄alkyl. In some embodiments of any one of the aspects described herein, R₁ is —CH₃. In some embodiments of any one of the aspects described herein, R₁ is —CH₂CH₃. In some embodiments of any one of the aspects described herein, R₁ is substituted or unsubstituted aryl. In some embodiments of any one of the aspects described herein, R₁ is unsubstituted phenyl. In some embodiments of any one of the aspects described herein, R₁ is substituted or unsubstituted C₁-C₈alkyl or substituted or unsubstituted phenyl. In some embodiments of any one of the aspects described herein, R₁ is H. In some embodiments of any one of the aspects described herein, R₁ is OH.

In some embodiments of any one of the aspects described herein, R₁′ is substituted or unsubstituted C₁-C₈alkyl. In some embodiments of any one of the aspects described herein, R₁′ is substituted C₁-C₈alkyl. In some embodiments of any one of the aspects described herein, R₁′ is —CF₃. In some embodiments of any one of the aspects described herein, R₁′ is unsubstituted C₁-C₈alkyl. In some embodiments of any one of the aspects described herein, R₁′ is unsubstituted C₁-C₄alkyl. In some embodiments of any one of the aspects described herein, R₁′ is —CH₃. In some embodiments of any one of the aspects described herein, R₁′ is —CH₂CH₃. In some embodiments of any one of the aspects described herein, R₁′ is substituted or unsubstituted aryl. In some embodiments of any one of the aspects described herein, R₁′ is unsubstituted phenyl. In some embodiments of any one of the aspects described herein, R₁′ is substituted or unsubstituted C₁-C₈alkyl or substituted or unsubstituted phenyl. In some embodiments of any one of the aspects described herein, R₁′ is H. In some embodiments of any one of the aspects described herein, R₁′ is OH.

In some embodiments of any one of the aspects described herein, one of R₁ and R₁′ is OH, e.g., only one of R₁ and R₁′ is OH. Accordingly, in some embodiments, R₁′ is OH. In some other embodiments, R₁ is OH.

In some embodiments of any one of the aspects described herein, R₁′ is OH and R₁ is substituted or unsubstituted C₁-C₈alkyl. In some embodiments of any one of the aspects described herein, R₁′ is OH and R₁ is substituted C₁-C₈alkyl. In some embodiments of any one of the aspects described herein, R₁′ is OH and R₁ is —CF₃. In some embodiments of any one of the aspects described herein, R₁′ is OH and R₁ is unsubstituted C₁-C₈alkyl. In some embodiments of any one of the aspects described herein, R₁′ is OH and R₁ is unsubstituted C₁-C₄alkyl. In some embodiments of any one of the aspects described herein, R₁′ is OH and R₁ is —CH₃. In some embodiments of any one of the aspects described herein, R₁′ is OH and R₁ is —CH₂CH₃. In some embodiments of any one of the aspects described herein, R₁′ is OH and R₁ is substituted or unsubstituted aryl. In some embodiments of any one of the aspects described herein, R₁′ is OH and R₁ is unsubstituted phenyl. In some embodiments of any one of the aspects described herein, R₁′ is OH and R₁ is or unsubstituted C₁-C₈alkyl or substituted or unsubstituted phenyl. In some embodiments of any one of the aspects described herein, R₁′ is OH and R₁ is H.

In some embodiments of any one of the aspects described herein, R₁ is OH and R₁′ is substituted or unsubstituted C₁-C₈alkyl. In some embodiments of any one of the aspects described herein, R₁ is OH and R₁′ is substituted C₁-C₈alkyl. In some embodiments of any one of the aspects described herein, R₁ is OH and R₁′ is —CF₃. In some embodiments of any one of the aspects described herein, R₁ is OH and R₁′ is unsubstituted C₁-C₈alkyl. In some embodiments of any one of the aspects described herein, R₁ is OH and R₁′ is unsubstituted C₁-C₄alkyl. In some embodiments of any one of the aspects described herein, R₁ is OH and R₁′ is —CH₃. In some embodiments of any one of the aspects described herein, R₁ is OH and R₁′ is —CH₂CH₃. In some embodiments of any one of the aspects described herein, R₁ is OH and R₁′ is substituted or unsubstituted aryl. In some embodiments of any one of the aspects described herein, R₁ is OH and R₁′ is unsubstituted phenyl. In some embodiments of any one of the aspects described herein, R₁ is OH and R₁′ is substituted or unsubstituted C₁-C₈alkyl or substituted or unsubstituted phenyl. In some embodiments of any one of the aspects described herein, R₁ is OH and R₁′ is H.

In compounds of any one of the aspects, R₂ can be hydrogen, deuterium, C₁-C₈alkyl, or —OH. For example, R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen, deuterium, or —OH. In some embodiments, R₂ is hydrogen. In some other embodiments, R₂ is deuterium. In still some other embodiments, R₂ is OH.

In compounds of any one of the aspects, R₃ can be hydrogen, deuterium, C₁-C₈alkyl, or —OH. For example, R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen, deuterium, or —OH. In some embodiments, R₃ is hydrogen. In some other embodiments, R₃ is deuterium. In still some other embodiments, R₃ is OH.

In compounds of any one of the aspects, R₄ can be hydrogen, deuterium, C₁-C₈alkyl, or —OH. For example, R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is hydrogen, deuterium, or —OH. In some embodiments, R₄ is hydrogen. In some other embodiments, R₄ is deuterium. In still some other embodiments, R₄ is OH.

In compounds of any one of the aspects, R₅ can be hydrogen, deuterium, C₁-C₈alkyl, or —OH. For example, R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₅ is hydrogen, deuterium, or —OH. In some embodiments, R₅ is hydrogen. In some other embodiments, R₅ is deuterium. In still some other embodiments, R₅ is OH.

In compounds of any one of the aspects, R₂ is hydrogen and R₃ can be hydrogen, deuterium, C₁-C₈alkyl, or —OH. For example, R₂ is hydrogen and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₃ is hydrogen, deuterium, or —OH. In some embodiments, R₂ is hydrogen and R₃ is hydrogen. In some other embodiments, R₂ is hydrogen and R₃ is deuterium. In still some other embodiments, R₂ is hydrogen and R₃ is OH.

In compounds of any one of the aspects, R₂ is deuterium and R₃ can be hydrogen, deuterium, C₁-C₈alkyl, or —OH. For example, R₂ is deuterium and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₃ is hydrogen, deuterium, or —OH. In some embodiments, R₂ is deuterium and R₃ is hydrogen. In some other embodiments, R₂ is deuterium and R₃ is deuterium. In still some other embodiments, R₂ is deuterium and R₃ is OH.

In compounds of any one of the aspects, R₂ is C₁-C₈alkyl and R₃ can be hydrogen, deuterium, C₁-C₈alkyl, or —OH. For example, R₂ is C₁-C₈alkyl and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₃ is hydrogen, deuterium, or —OH. In some embodiments, R₂ is C₁-C₈alkyl and R₃ is hydrogen. In some other embodiments, R₂ is C₁-C₈alkyl and R₃ is deuterium. In still some other embodiments, R₂ is C₁-C₈alkyl and R₃ is OH.

In compounds of any one of the aspects, R₂ is OH and R₃ can be hydrogen, deuterium, C₁-C₈alkyl, or —OH. For example, R₂ is OH and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₃ is hydrogen, deuterium, or —OH. In some embodiments, R₂ is OH and R₃ is hydrogen. In some other embodiments, R₂ is OH and R₃ is deuterium. In still some other embodiments, R₂ is OH and R₃ is OH.

In compounds of any one of the aspects, R₂ is hydrogen and R₄ can be hydrogen, deuterium, C₁-C₈alkyl, or —OH. For example, R₂ is hydrogen and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₄ is hydrogen, deuterium, or —OH. In some embodiments, R₂ is hydrogen and R₄ is hydrogen. In some other embodiments, R₂ is hydrogen and R₄ is deuterium. In still some other embodiments, R₂ is hydrogen and R₄ is OH.

In compounds of any one of the aspects, R₂ is deuterium and R₄ can be hydrogen, deuterium, C₁-C₈alkyl, or —OH. For example, R₂ is deuterium and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₄ is hydrogen, deuterium, or —OH. In some embodiments, R₂ is deuterium and R₄ is hydrogen. In some other embodiments, R₂ is deuterium and R₄ is deuterium. In still some other embodiments, R₂ is deuterium and R₄ is OH.

In compounds of any one of the aspects, R₂ is C₁-C₈alkyl and R₄ can be hydrogen, deuterium, C₁-C₈alkyl, or —OH. For example, R₂ is C₁-C₈alkyl and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₄ is hydrogen, deuterium, or —OH. In some embodiments, R₂ is C₁-C₈alkyl and R₄ is hydrogen. In some other embodiments, R₂ is C₁-C₈alkyl and R₄ is deuterium. In still some other embodiments, R₂ is C₁-C₈alkyl and R₄ is OH.

In compounds of any one of the aspects, R₂ is OH and R₄ can be hydrogen, deuterium, C₁-C₈alkyl, or —OH. For example, R₂ is OH and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₄ is hydrogen, deuterium, or —OH. In some embodiments, R₂ is OH and R₄ is hydrogen. In some other embodiments, R₂ is OH and R₄ is deuterium. In still some other embodiments, R₂ is OH and R₄ is OH.

In compounds of any one of the aspects, R₂ is hydrogen and R₅ can be hydrogen, deuterium, C₁-C₈alkyl, or —OH. For example, R₂ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₅ is hydrogen, deuterium, or —OH. In some embodiments, R₂ is hydrogen and R₅ is hydrogen. In some other embodiments, R₂ is hydrogen and R₅ is deuterium. In still some other embodiments, R₂ is hydrogen and R₅ is OH.

In compounds of any one of the aspects, R₂ is deuterium and R₅ can be hydrogen, deuterium, C₁-C₈alkyl, or —OH. For example, R₂ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₅ is hydrogen, deuterium, or —OH. In some embodiments, R₂ is deuterium and R₅ is hydrogen. In some other embodiments, R₂ is deuterium and R₅ is deuterium. In still some other embodiments, R₂ is deuterium and R₅ is OH.

In compounds of any one of the aspects, R₂ is C₁-C₈alkyl and R₅ can be hydrogen, deuterium, C₁-C₈alkyl, or —OH. For example, R₂ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₅ is hydrogen, deuterium, or —OH. In some embodiments, R₂ is C₁-C₈alkyl and R₅ is hydrogen. In some other embodiments, R₂ is C₁-C₈alkyl and R₅ is deuterium. In still some other embodiments, R₂ is C₁-C₈alkyl and R₅ is OH.

In compounds of any one of the aspects, R₂ is OH and R₅ can be hydrogen, deuterium, C₁-C₈alkyl, or —OH. For example, R₂ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₅ is hydrogen, deuterium, or —OH. In some embodiments, R₂ is OH and R₅ is hydrogen. In some other embodiments, R₂ is OH and R₅ is deuterium. In still some other embodiments, R₂ is OH and R₅ is OH.

In compounds of any one of the aspects, R₃ is hydrogen and R₄ can be hydrogen, deuterium, C₁-C₈alkyl, or —OH. For example, R₃ is hydrogen and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen and R₄ is hydrogen, deuterium, or —OH. In some embodiments, R₃ is hydrogen and R₄ is hydrogen. In some other embodiments, R₃ is hydrogen and R₄ is deuterium. In still some other embodiments, R₃ is hydrogen and R₄ is OH.

In compounds of any one of the aspects, R₃ is deuterium and R₄ can be hydrogen, deuterium, C₁-C₈alkyl, or —OH. For example, R₃ is deuterium and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is deuterium and R₄ is hydrogen, deuterium, or —OH. In some embodiments, R₃ is deuterium and R₄ is hydrogen. In some other embodiments, R₃ is deuterium and R₄ is deuterium. In still some other embodiments, R₃ is deuterium and R₄ is OH.

In compounds of any one of the aspects, R₃ is C₁-C₈alkyl and R₄ can be hydrogen, deuterium, C₁-C₈alkyl, or —OH. For example, R₃ is C₁-C₈alkyl and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is C₁-C₈alkyl and R₄ is hydrogen, deuterium, or —OH. In some embodiments, R₃ is C₁-C₈alkyl and R₄ is hydrogen. In some other embodiments, R₃ is C₁-C₈alkyl and R₄ is deuterium. In still some other embodiments, R₃ is C₁-C₈alkyl and R₄ is OH.

In compounds of any one of the aspects, R₃ is OH and R₄ can be hydrogen, deuterium, C₁-C₈alkyl, or —OH. For example, R₃ is OH and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is OH and R₄ is hydrogen, deuterium, or —OH. In some embodiments, R₃ is OH and R₄ is hydrogen. In some other embodiments, R₃ is OH and R₄ is deuterium. In still some other embodiments, R₃ is OH and R₄ is OH.

In compounds of any one of the aspects, R₃ is hydrogen and R₅ can be hydrogen, deuterium, C₁-C₈alkyl, or —OH. For example, R₃ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen and R₅ is hydrogen, deuterium, or —OH. In some embodiments, R₃ is hydrogen and R₅ is hydrogen. In some other embodiments, R₃ is hydrogen and R₅ is deuterium. In still some other embodiments, R₃ is hydrogen and R₅ is OH.

In compounds of any one of the aspects, R₃ is deuterium and R₅ can be hydrogen, deuterium, C₁-C₈alkyl, or —OH. For example, R₃ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is deuterium and R₅ is hydrogen, deuterium, or —OH. In some embodiments, R₃ is deuterium and R₅ is hydrogen. In some other embodiments, R₃ is deuterium and R₅ is deuterium. In still some other embodiments, R₃ is deuterium and R₅ is OH.

In compounds of any one of the aspects, R₃ is C₁-C₈alkyl and R₅ can be hydrogen, deuterium, C₁-C₈alkyl, or —OH. For example, R₃ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is C₁-C₈alkyl and R₅ is hydrogen, deuterium, or —OH. In some embodiments, R₃ is C₁-C₈alkyl and R₅ is hydrogen. In some other embodiments, R₃ is C₁-C₈alkyl and R₅ is deuterium. In still some other embodiments, R₃ is C₁-C₈alkyl and R₅ is OH.

In compounds of any one of the aspects, R₃ is OH and R₅ can be hydrogen, deuterium, C₁-C₈alkyl, or —OH. For example, R₃ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is OH and R₅ is hydrogen, deuterium, or —OH. In some embodiments, R₃ is OH and R₅ is hydrogen. In some other embodiments, R₃ is OH and R₅ is deuterium. In still some other embodiments, R₃ is OH and R₅ is OH.

In compounds of any one of the aspects, R₄ is hydrogen and R₅ can be hydrogen, deuterium, C₁-C₈alkyl, or —OH. For example, R₄ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is hydrogen and R₅ is hydrogen, deuterium, or —OH. In some embodiments, R₄ is hydrogen and R₅ is hydrogen. In some other embodiments, R₄ is hydrogen and R₅ is deuterium. In still some other embodiments, R₄ is hydrogen and R₅ is OH.

In compounds of any one of the aspects, R₄ is deuterium and R₅ can be hydrogen, deuterium, C₁-C₈alkyl, or —OH. For example, R₄ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is deuterium and R₅ is hydrogen, deuterium, or —OH. In some embodiments, R₄ is deuterium and R₅ is hydrogen. In some other embodiments, R₄ is deuterium and R₅ is deuterium. In still some other embodiments, R₄ is deuterium and R₅ is OH.

In compounds of any one of the aspects, R₄ is C₁-C₈alkyl and R₅ can be hydrogen, deuterium, C₁-C₈alkyl, or —OH. For example, R₄ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is C₁-C₈alkyl and R₅ is hydrogen, deuterium, or —OH. In some embodiments, R₄ is C₁-C₈alkyl and R₅ is hydrogen. In some other embodiments, R₄ is C₁-C₈alkyl and R₅ is deuterium. In still some other embodiments, R₄ is C₁-C₈alkyl and R₅ is OH.

In compounds of any one of the aspects, R₄ is OH and R₅ can be hydrogen, deuterium, C₁-C₈alkyl, or —OH. For example, R₄ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is OH and R₅ is hydrogen, deuterium, or —OH. In some embodiments, R₄ is OH and R₅ is hydrogen. In some other embodiments, R₄ is OH and R₅ is deuterium. In still some other embodiments, R₄ is OH and R₅ is OH.

In some embodiments of any one of the aspects, R₂, R₃, R₄, and R₅ are each deuterium. In some embodiments of any one of the aspects, R₂, R₃, R₄, and R₅ are each hydrogen. In some embodiments of any one of the aspects, R₂ is —OH, and R₃, R₄, and R₅ are each hydrogen. In some embodiments of any one of the aspects, R₃ is —OH, and R₂, R₄, and R₅ are each hydrogen. In some embodiments of any one of the aspects, R₂ and R₄ are each —OH, and R₃ and R₅ are each hydrogen. In some embodiments of any one of the aspects, R₂ and R₅ are each —OH, and R₃ and R₄ are each hydrogen. In some embodiments of any one of the aspects, R₃ and R₄ are each —OH, and R₂ and R₅ are each hydrogen. In some embodiments of any one of the aspects, R₃ and R₅ are each —OH, and R₂ and R₄ are each hydrogen.

In some embodiments of any one of the aspects described herein, R₂ and R₄ form a second bond between the carbon atoms they are attached to. In some compounds of the various aspects described herein, R₂ and R₅ form a second bond between the carbon atoms they are attached to.

In compounds of any one of the aspects, R₂ and R₄ form a second bond between the carbon atoms they are attached to and R₃ can be hydrogen, deuterium, C₁-C₈alkyl, or —OH. For example, R₂ and R₄ form a second bond between the carbon atoms they are attached to and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₄ form a second bond between the carbon atoms they are attached to and R₃ is hydrogen, deuterium, or —OH. In some embodiments, R₂ and R₄ form a second bond between the carbon atoms they are attached to and R₃ is hydrogen. In some other embodiments, R₂ and R₄ form a second bond between the carbon atoms they are attached to and R₃ is deuterium. In still some other embodiments, R₂ and R₄ form a second bond between the carbon atoms they are attached to and R₃ is OH.

In compounds of any one of the aspects, R₂ and R₄ form a second bond between the carbon atoms they are attached to and R₅ can be hydrogen, deuterium, C₁-C₈alkyl, or —OH. For example, R₂ and R₄ form a second bond between the carbon atoms they are attached to and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₄ form a second bond between the carbon atoms they are attached to and R₅ is hydrogen, deuterium, or —OH. In some embodiments, R₂ and R₄ form a second bond between the carbon atoms they are attached to and R₅ is hydrogen. In some other embodiments, R₂ and R₄ form a second bond between the carbon atoms they are attached to and R₅ is deuterium. In still some other embodiments, R₂ and R₄ form a second bond between the carbon atoms they are attached to and R₅ is OH.

In compounds of any one of the aspects, R₂ and R₅ form a second bond between the carbon atoms they are attached to and R₃ can be hydrogen, deuterium, C₁-C₈alkyl, or —OH. For example, R₂ and R₅ form a second bond between the carbon atoms they are attached to and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₅ form a second bond between the carbon atoms they are attached to and R₃ is hydrogen, deuterium, or —OH. In some embodiments, R₂ and R₅ form a second bond between the carbon atoms they are attached to and R₃ is hydrogen. In some other embodiments, R₂ and R₅ form a second bond between the carbon atoms they are attached to and R₃ is deuterium. In still some other embodiments, R₂ and R₅ form a second bond between the carbon atoms they are attached to and R₃ is OH.

In compounds of any one of the aspects, R₂ and R₅ form a second bond between the carbon atoms they are attached to and R₄ can be hydrogen, deuterium, C₁-C₈alkyl, or —OH. For example, R₂ and R₅ form a second bond between the carbon atoms they are attached to and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₅ form a second bond between the carbon atoms they are attached to and R₄ is hydrogen, deuterium, or —OH. In some embodiments, R₂ and R₅ form a second bond between the carbon atoms they are attached to and R₄ is hydrogen. In some other embodiments, R₂ and R₅ form a second bond between the carbon atoms they are attached to and R₄ is deuterium. In still some other embodiments, R₂ and R₅ form a second bond between the carbon atoms they are attached to and R₄ is OH.

In some embodiments of any one of the aspects described herein, R₃ and R₄ form a second bond between the carbon atoms they are attached to. In some compounds of the various aspects described herein, R₃ and R₅ form a second bond between the carbon atoms they are attached to.

In compounds of any one of the aspects, R₃ and R₄ form a second bond between the carbon atoms they are attached to and R₂ can be hydrogen, deuterium, C₁-C₈alkyl, or —OH. For example, R₃ and R₄ form a second bond between the carbon atoms they are attached to and R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₄ form a second bond between the carbon atoms they are attached to and R₂ is hydrogen, deuterium, or —OH. In some embodiments, R₃ and R₄ form a second bond between the carbon atoms they are attached to and R₂ is hydrogen.

In some other embodiments, R₃ and R₄ form a second bond between the carbon atoms they are attached to and R₂ is deuterium. In still some other embodiments, R₃ and R₄ form a second bond between the carbon atoms they are attached to and R₂ is OH.

In compounds of any one of the aspects, R₃ and R₄ form a second bond between the carbon atoms they are attached to and R₅ can be hydrogen, deuterium, C₁-C₈alkyl, or —OH. For example, R₃ and R₄ form a second bond between the carbon atoms they are attached to and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₄ form a second bond between the carbon atoms they are attached to and R₅ is hydrogen, deuterium, or —OH. In some embodiments, R₃ and R₄ form a second bond between the carbon atoms they are attached to and R₅ is hydrogen. In some other embodiments, R₃ and R₄ form a second bond between the carbon atoms they are attached to and R₅ is deuterium. In still some other embodiments, R₃ and R₄ form a second bond between the carbon atoms they are attached to and R₅ is OH.

In compounds of any one of the aspects, R₃ and R₅ form a second bond between the carbon atoms they are attached to and R₂ can be hydrogen, deuterium, C₁-C₈alkyl, or —OH. For example, R₃ and R₅ form a second bond between the carbon atoms they are attached to and R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₅ form a second bond between the carbon atoms they are attached to and R₂ is hydrogen, deuterium, or —OH. In some embodiments, R₃ and R₅ form a second bond between the carbon atoms they are attached to and R₂ is hydrogen. In some other embodiments, R₃ and R₅ form a second bond between the carbon atoms they are attached to and R₂ is deuterium. In still some other embodiments, R₃ and R₅ form a second bond between the carbon atoms they are attached to and R₂ is OH.

In compounds of any one of the aspects, R₃ and R₅ form a second bond between the carbon atoms they are attached to and R₄ can be hydrogen, deuterium, C₁-C₈alkyl, or —OH. For example, R₄ and R₅ form a second bond between the carbon atoms they are attached to and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₅ form a second bond between the carbon atoms they are attached to and R₄ is hydrogen, deuterium, or —OH. In some embodiments, R₃ and R₅ form a second bond between the carbon atoms they are attached to and R₄ is hydrogen. In some other embodiments, R₃ and R₅ form a second bond between the carbon atoms they are attached to and R₄ is deuterium. In still some other embodiments, R₃ and R₅ form a second bond between the carbon atoms they are attached to and R₄ is OH.

In some embodiments of any one of the aspects, R₆ is substituted or unsubstituted aryl. In some embodiments of any one of the aspects, R₆ is substituted or unsubstituted phenyl. In some embodiments of any one of the aspects, R₆ is unsubstituted phenyl. In some embodiments of any one of the aspects, R₆ is substituted phenyl. In some embodiments of any one of the aspects, R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments of any one of the aspects, R₆ is phenyl substituted with at least one substituent selected from alkyl, hydroxy, alkoxy, halogen, and haloalkyl. In some embodiments of any one of the aspects, R₆ is phenyl substituted with at least one halogen substituent. In some embodiments of any one of the aspects, R₆ is phenyl substituted with at least one fluoro substituent. In some embodiments of any one of the aspects, R₆ is 4-fluorophenyl.

In some embodiments of any one of the aspects, R₆ is substituted or unsubstituted heteroaryl. In some embodiments of any one of the aspects, R₆ is unsubstituted heteroaryl. In some embodiments of any one of the aspects, R₆ is substituted heteroaryl. In some embodiments of any one of the aspects, R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments of any one of the aspects, R₆ is heteroaryl substituted with at least one substituent selected from alkyl, hydroxy, alkoxy, halogen, and haloalkyl. In some embodiments of any one of the aspects, R₆ is heteroaryl substituted with at least one halogen substituent. In some embodiments of any one of the aspects, R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments of any one of the aspects described herein, R₆ is C₆-C₁₀aryl optionally substituted with 1, 2, 3, or 4 R₉ groups. In some embodiments of any one of the aspects described herein, R₆ is C₆-C₁₀aryl substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is halogen. In some embodiments of any one of the aspects described herein, R₆ is phenyl substituted with 1 R₉ group and R₉ is fluoro. In some embodiments of any one of the aspects described herein, R₆ is phenyl substituted with 1 R₉ group and R₉ is C₁₋₆alkyl optionally substituted with one, two, or three groups independently selected from halogen. In some embodiments of any one of the aspects described herein, R₆ is phenyl substituted with 1 R₉ group and R₉ is unsubstituted C₁₋₆alkyl. In some embodiments of any one of the aspects described herein, R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is unsubstituted phenyl.

In some embodiments of any one of the aspects described herein, R₆ is C₂-C₉heteroaryl optionally substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is halogen. In some embodiments of any one of the aspects described herein, R₆ is C₂-C₉heteroaryl substituted with 1 R₉ group and R₉ is fluoro. In some embodiments of any one of the aspects described herein, R₆ is C₂-C₉heteroaryl substituted with 1 R₉ group and R₉ is C₁₋₆alkyl optionally substituted with one, two, or three groups independently selected from halogen. In some embodiments of any one of the aspects described herein, R₆ is C₂-C₉heteroaryl substituted with 1 R₉ group and R₉ is unsubstituted C₁₋₆alkyl. In some embodiments of any one of the aspects described herein, R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is unsubstituted phenyl.

In some embodiments of any one of the aspects described herein, R₆ is unsubstituted C₂-C₉heteroaryl.

In some embodiments of any one of the aspects described herein, R₆ is pyridyl optionally substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is halogen. In some embodiments of any one of the aspects described herein, R₆ is pyridyl substituted with 1 R₉ group and R₉ is fluoro. In some embodiments of any one of the aspects described herein, R₆ is pyridyl substituted with 1 R₉ group and R₉ is C₁₋₆alkyl optionally substituted with one, two, or three groups independently selected from halogen. In some embodiments of any one of the aspects described herein, R₆ is pyridyl substituted with 1 R₉ group and R₉ is unsubstituted C₁₋₆alkyl. In some embodiments of any one of the aspects described herein, R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is unsubstituted phenyl. In some embodiments of any one of the aspects described herein, R₆ is unsubstituted pyridyl.

In some embodiments of any one of the aspects, R₆ is a substituted or unsubstituted alkyl. For example, R₆ is a substituted or unsubstituted C₁-C₆alkyl. In some embodiments of any one of the aspects, R₆ can be methyl, ethyl, propyl, i-propyl, n-butyl, t-butyl, n-pentyl or hexyl. For example, R₆ can be n-butyl or t-butyl. In some embodiments of any one of the aspects, R₆ is n-butyl. In some other embodiments of any one of the aspects, R₆ is t-butyl.

In compounds of the any one of the aspects described herein, R₇ can be H, substituted or unsubstituted alkyl, or —C(O)NR₁₀R₁₁. For example, R₇ can be hydrogen or substituted or unsubstituted C₁-C₈alkyl. In some embodiments of any one of the aspects, R₇ is hydrogen. In some embodiments of any one of the aspects, R₇ is substituted or unsubstituted C₁-C₈alkyl. In some embodiments of any one of the aspects, R₇ is unsubstituted C₁-C₈alkyl. In some embodiments of any one of the aspects, R₇ is —CH₃. In some embodiments of any one of the aspects, R₇ is C(O)NR₁₀R₁₁. In some embodiments of any one of the aspects, R₇ is substituted or unsubstituted C₁-C₈alkyl. In some embodiments of any one of the aspects, R₇ is —C(O)NR₁₀R₁₁, and R₁₀ and R₁₁ are independently substituted or unsubstituted C₁-C₈alkyl. In some embodiments of any one of the aspects, R₇ is —C(O)NR₁₀R₁₁, and R₁₀ and R₁₁ are each CH₃. In some embodiments of any one of the aspects, R₇ is —C(O)NR₁₀R₁₁, R₁₀ is hydrogen, and R₁₁ is substituted or unsubstituted C₁-C₈alkyl. In some embodiments of any one of the aspects, R₇ is —C(O)NR₁₀R₁₁, R₁₀ is hydrogen, and R₁₁ is —CH₃. In some embodiments of any one of the aspects, R₇ is —C(O)NR₁₀R₁₁, R₁₀ is substituted or unsubstituted aryl, and R₁₁ is substituted or unsubstituted C₁-C₈alkyl. In some embodiments of any one of the aspects, R₇ is —C(O)NR₁₀R₁₁, R₁₀ is substituted or unsubstituted aryl, and R₁₁ is hydrogen.

In the compounds described herein, R₈ can be H or OH. For example, in some embodiments of any one of the aspects, R₈ is H. In some other embodiments of any one of the aspects, R₈ is OH.

In some embodiments of any one of the aspects described herein, the compound of Formula (I) is of Formula (Ia):

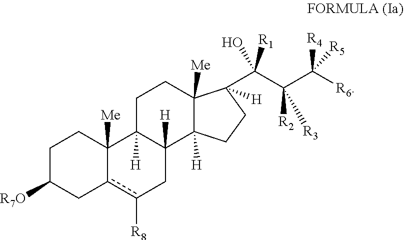

In some embodiments of any one of the aspects described herein, the compound of Formula (I) is of Formula (Ib):

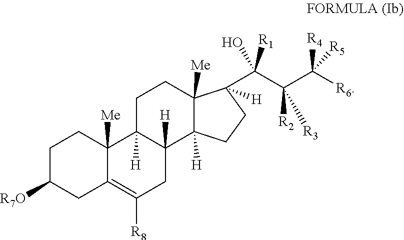

In some embodiments of any one of the aspects described herein, the compound of Formula (I) is of Formula (Ic):

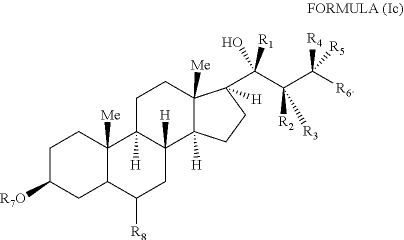

In some embodiments of any one of the aspects described herein, the compound of Formula (I) is of Formula (II):

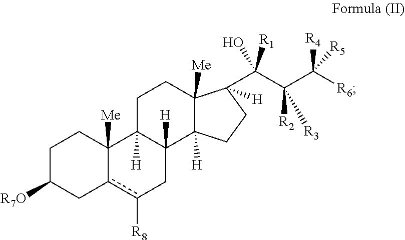

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl; [symbol] is a single or double bond;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (II) wherein, [symbol] is a single bond. In some embodiments is a compound of Formula (II) wherein [symbol] is a double bond.

In some embodiments is a compound of Formula (II), wherein R₆ is substituted or unsubstituted aryl. In some embodiments is a compound of Formula (II), wherein R₆ is substituted or unsubstituted phenyl. In some embodiments is a compound of Formula (II), wherein R₆ is unsubstituted phenyl. In some embodiments is a compound of Formula (II), wherein R₆ is substituted phenyl. In some embodiments is a compound of Formula (II), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (II), wherein R₆ is phenyl substituted with at least one substituent selected from alkyl, hydroxy, alkoxy, halogen, and haloalkyl. In some embodiments is a compound of Formula (II), wherein R₆ is phenyl substituted with at least one halogen substituent. In some embodiments is a compound of Formula (II), wherein R₆ is phenyl substituted with at least one fluoro substituent. In some embodiments is a compound of Formula (II), wherein R₆ is 4-fluorophenyl.

In some embodiments is a compound of Formula (II), wherein R₆ is substituted or unsubstituted heteroaryl. In some embodiments is a compound of Formula (II), wherein R₆ is unsubstituted heteroaryl. In some embodiments is a compound of Formula (II), wherein R₆ is substituted heteroaryl. In some embodiments is a compound of Formula (II), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (II), wherein R₆ is heteroaryl substituted with at least one substituent selected from alkyl, hydroxy, alkoxy, halogen, and haloalkyl. In some embodiments is a compound of Formula (II), wherein R₆ is heteroaryl substituted with at least one halogen substituent. In some embodiments is a compound of Formula (II), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (II), wherein R₂, R₃, R₄, and R₅ are independently hydrogen or deuterium. In some embodiments is a compound of Formula (II), wherein R₂, R₃, R₄, and R₅ are each deuterium. In some embodiments is a compound of Formula (II), wherein R₂, R₃, R₄, and R₅ are each hydrogen. In some embodiments is a compound of Formula (II), wherein R₂ is —OH, and R₃, R₄, and R₅ are each hydrogen. In some embodiments is a compound of Formula (II), wherein R₃ is —OH, and R₂, R₄, and R₅ are each hydrogen. In some embodiments is a compound of Formula (II), wherein R₂ and R₄ are each —OH, and R₃ and R₅ are each hydrogen. In some embodiments is a compound of Formula (II), wherein R₂ and R₅ are each —OH, and R₃ and R₄ are each hydrogen. In some embodiments is a compound of Formula (II), wherein R₃ and R₄ are each —OH, and R₂ and R₅ are each hydrogen. In some embodiments is a compound of Formula (II), wherein R₃ and R₅ are each —OH, and R₂ and R₄ are each hydrogen.

In some embodiments is a compound of Formula (II), wherein R₁ is substituted or unsubstituted C₁-C₈alkyl. In some embodiments is a compound of Formula (II), wherein R₁ is substituted C₁-C₈alkyl. In some embodiments is a compound of Formula (II), wherein R₁ is —CF₃. In some embodiments is a compound of Formula (II), wherein R₁ is unsubstituted C₁-C₈alkyl. In some embodiments is a compound of Formula (II), wherein R₁ is unsubstituted C₁-C₄alkyl. In some embodiments is a compound of Formula (II), wherein R₁ is —CH₃. In some embodiments is a compound of Formula (II), wherein R₁ is —CH₂CH₃. In some embodiments is a compound of Formula (II), wherein R₁ is substituted or unsubstituted aryl. In some embodiments is a compound of Formula (II), wherein R₁ is unsubstituted phenyl. In some embodiments is a compound of Formula (II), wherein R₁ is substituted or unsubstituted C₁-C₈alkyl or substituted or unsubstituted phenyl.

In some embodiments is a compound of Formula (II), wherein R₇ is hydrogen or substituted or unsubstituted C₁-C₈alkyl. In some embodiments is a compound of Formula (II), wherein R₇ is hydrogen. In some embodiments is a compound of Formula (II), wherein R₇ is substituted or unsubstituted C₁-C₈alkyl. In some embodiments is a compound of Formula (II), wherein R₇ is unsubstituted C₁-C₈alkyl. In some embodiments is a compound of Formula (II), wherein R₇ is —CH₃. In some embodiments is a compound of Formula (II), wherein R₇ is C(O)NR₁₀R₁₁. In some embodiments is a compound of Formula (II), wherein R₇ is substituted or unsubstituted C₁-C₈alkyl. In some embodiments is a compound of Formula (II), wherein R₇ is C(O)NR₁₀R₁₁, and R₁₀ and R₁₁ are independently substituted or unsubstituted C₁-C₈alkyl. In some embodiments is a compound of Formula (II), wherein R₇ is —C(O)NR₁₀R₁₁, and R₁₀ and R₁₁ are each CH₃. In some embodiments is a compound of Formula (II), wherein R₇ is —C(O)NR₁₀R₁₁, R₁₀ is hydrogen, and R₁ is substituted or unsubstituted C₁-C₈alkyl. In some embodiments is a compound of Formula (II), wherein R₇ is —C(O)NR₁₀R₁₁, R₁₀ is hydrogen, and R₁₁ is —CH₃. In some embodiments is a compound of Formula (II), wherein R₇ is —C(O)NR₁₀R₁₁, R₁₀ is substituted or unsubstituted aryl, and R₁₁ is substituted or unsubstituted C₁-C₈alkyl. In some embodiments is a compound of Formula (II), wherein R₇ is —C(O)NR₁₀R₁₁, R₁₀ is substituted or unsubstituted aryl, and R₁ is hydrogen.

In some embodiments of any one of the aspects described herein, the compound of Formula (I) is of Formula (IIa):

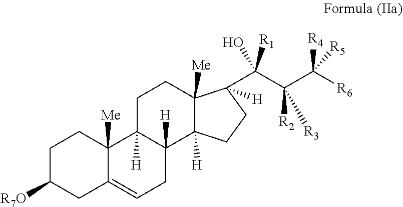

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (IIa), wherein R₆ is substituted or unsubstituted aryl. In some embodiments is a compound of Formula (IIa), wherein R₆ is substituted or unsubstituted phenyl. In some embodiments is a compound of Formula (IIa), wherein R₆ is unsubstituted phenyl. In some embodiments is a compound of Formula (IIa), wherein R₆ is substituted phenyl. In some embodiments is a compound of Formula (IIa), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIa), wherein R₆ is phenyl substituted with at least one substituent selected from alkyl, hydroxy, alkoxy, halogen, and haloalkyl. In some embodiments is a compound of Formula (IIa), wherein R₆ is phenyl substituted with at least one halogen substituent. In some embodiments is a compound of Formula (IIa), wherein R₆ is phenyl substituted with at least one fluoro substituent.

In some embodiments is a compound of Formula (IIa), wherein R₆ is substituted or unsubstituted heteroaryl. In some embodiments is a compound of Formula (IIa), wherein R₆ is unsubstituted heteroaryl. In some embodiments is a compound of Formula (IIa), wherein R₆ is substituted heteroaryl. In some embodiments is a compound of Formula (IIa), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIa), wherein R₆ is heteroaryl substituted with at least one substituent selected from alkyl, hydroxy, alkoxy, halogen, and haloalkyl. In some embodiments is a compound of Formula (IIa), wherein R₆ is heteroaryl substituted with at least one halogen substituent. In some embodiments is a compound of Formula (IIa), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (IIa), wherein R₂, R₃, R₄, and R₅ are each deuterium. In some embodiments is a compound of Formula (IIa), wherein R₂, R₃, R₄, and R₅ are each hydrogen. In some embodiments is a compound of Formula (IIa), wherein R₂ is —OH, and R₃, R₄, and R₅ are each hydrogen. In some embodiments is a compound of Formula (IIa), wherein R₃ is —OH, and R₂, R₄, and R₅ are each hydrogen. In some embodiments is a compound of Formula (IIa), wherein R₂ and R₄ are each —OH, and R₃ and R₅ are each hydrogen. In some embodiments is a compound of Formula (IIa), wherein R₂ and R₅ are each —OH, and R₃ and R₄ are each hydrogen. In some embodiments is a compound of Formula (IIa), wherein R₃ and R₄ are each —OH, and R₂ and R₅ are each hydrogen. In some embodiments is a compound of Formula (IIa), wherein R₃ and R₅ are each —OH, and R₂ and R₄ are each hydrogen.

In some embodiments is a compound of Formula (IIa), wherein R₁ is substituted or unsubstituted C₁-C₈alkyl. In some embodiments is a compound of Formula (IIa), wherein R₁ is substituted C₁-C₈alkyl. In some embodiments is a compound of Formula (IIa), wherein R₁ is —CF₃. In some embodiments is a compound of Formula (IIa), wherein R₁ is unsubstituted C₁-C₈alkyl. In some embodiments is a compound of Formula (IIa), wherein R₁ is —CH₃. In some embodiments is a compound of Formula (IIa), wherein R₁ is —CH₂CH₃. In some embodiments is a compound of Formula (IIa), wherein R₁ is substituted or unsubstituted aryl. In some embodiments is a compound of Formula (IIa), wherein R₁ is unsubstituted phenyl.

In some embodiments is a compound of Formula (IIa), wherein R₇ is hydrogen. In some embodiments is a compound of Formula (IIa), wherein R₇ is substituted or unsubstituted C₁-C₈alkyl. In some embodiments is a compound of Formula (IIa), wherein R₇ is —CH₃. In some embodiments is a compound of Formula (IIa), wherein R₇ is —C(O)NR₁₀R₁₁. In some embodiments is a compound of Formula (IIa), wherein R₇ is substituted or unsubstituted C₁-C₈alkyl. In some embodiments is a compound of Formula (IIa), wherein R₇ is —C(O)NR₁₀R₁₁, and R₁₀ and R₁₁ are independently substituted or unsubstituted C₁-C₈alkyl. In some embodiments is a compound of Formula (IIa), wherein R₇ is —C(O)NR₁₀R₁₁, and R₁₀ and R₁₁ are each CH₃. In some embodiments is a compound of Formula (IIa), wherein R₇ is —C(O)NR₁₀R₁₁, R₁₀ is hydrogen, and R₁ is substituted or unsubstituted C₁-C₈alkyl. In some embodiments is a compound of Formula (IIa), wherein R₇ is —C(O)NR₁₀R₁₁, R₁₀ is hydrogen, and R₁₁ is —CH₃. In some embodiments is a compound of Formula (IIa), wherein R₇ is —C(O)NR₁₀R₁₁, R₁₀ is substituted or unsubstituted aryl, and R₁₁ is substituted or unsubstituted C₁-C₈alkyl. In some embodiments is a compound of Formula (IIa), wherein R₇ is —C(O)NR₁₀R₁₁, R₁₀ is substituted or unsubstituted aryl, and R₁₁ is hydrogen.

In some embodiments of any one of the aspects described herein, the compound of Formula (I) is a compound of Formula (IIb):

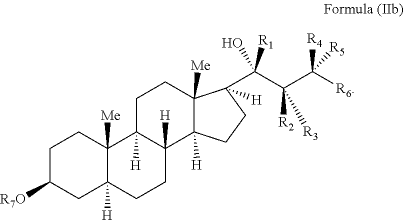

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (IIb), wherein R₆ is substituted or unsubstituted aryl. In some embodiments is a compound of Formula (IIb), wherein R₆ is substituted or unsubstituted phenyl. In some embodiments is a compound of Formula (IIb), wherein R₆ is unsubstituted phenyl. In some embodiments is a compound of Formula (IIb), wherein R₆ is substituted phenyl. In some embodiments is a compound of Formula (IIb), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIb), wherein R₆ is phenyl substituted with at least one substituent selected from alkyl, hydroxy, alkoxy, halogen, and haloalkyl. In some embodiments is a compound of Formula (IIb), wherein R₆ is phenyl substituted with at least one halogen substituent. In some embodiments is a compound of Formula (IIb), wherein R₆ is phenyl substituted with at least one fluoro substituent.

In some embodiments is a compound of Formula (IIb), wherein R₆ is substituted or unsubstituted heteroaryl. In some embodiments is a compound of Formula (IIb), wherein R₆ is unsubstituted heteroaryl. In some embodiments is a compound of Formula (IIb), wherein R₆ is substituted heteroaryl. In some embodiments is a compound of Formula (IIb), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIb), wherein R₆ is heteroaryl substituted with at least one substituent selected from alkyl, hydroxy, alkoxy, halogen, and haloalkyl. In some embodiments is a compound of Formula (IIb), wherein R₆ is heteroaryl substituted with at least one halogen substituent. In some embodiments is a compound of Formula (IIb), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (IIb), wherein R₂, R₃, R₄, and R₅ are each deuterium. In some embodiments is a compound of Formula (IIb), wherein R₂, R₃, R₄, and R₅ are each hydrogen. In some embodiments is a compound of Formula (IIb), wherein R₂ is —OH, and R₃, R₄, and R₅ are each hydrogen. In some embodiments is a compound of Formula (IIb), wherein R₃ is —OH, and R₂, R₄, and R₅ are each hydrogen. In some embodiments is a compound of Formula (IIb), wherein R₂ and R₄ are each —OH, and R₃ and R₅ are each hydrogen. In some embodiments is a compound of Formula (IIb), wherein R₂ and R₅ are each —OH, and R₃ and R₄ are each hydrogen. In some embodiments is a compound of Formula (IIb), wherein R₃ and R₄ are each —OH, and R₂ and R₅ are each hydrogen. In some embodiments is a compound of Formula (IIb), wherein R₃ and R₅ are each —OH, and R₂ and R₄ are each hydrogen.

In some embodiments is a compound of Formula (IIb), wherein R₁ is substituted or unsubstituted C₁-C₈alkyl. In some embodiments is a compound of Formula (IIb), wherein R₁ is substituted C₁-C₈alkyl. In some embodiments is a compound of Formula (IIb), wherein R₁ is —CF₃. In some embodiments is a compound of Formula (IIb), wherein R₁ is unsubstituted C₁-C₈alkyl. In some embodiments is a compound of Formula (IIb), wherein R₁ is —CH₃. In some embodiments is a compound of Formula (IIb), wherein R₁ is —CH₂CH₃. In some embodiments is a compound of Formula (IIb), wherein R₁ is substituted or unsubstituted aryl. In some embodiments is a compound of Formula (IIb), wherein R₁ is unsubstituted phenyl.

In some embodiments is a compound of Formula (IIb), wherein R₇ is hydrogen. In some embodiments is a compound of Formula (IIb), wherein R₇ is substituted or unsubstituted C₁-C₈alkyl. In some embodiments is a compound of Formula (IIb), wherein R₇ is —CH₃. In some embodiments is a compound of Formula (IIb), wherein R₇ is —C(O)NR₁₀R₁₁. In some embodiments is a compound of Formula (IIb), wherein R₇ is substituted or unsubstituted C₁-C₈alkyl. In some embodiments is a compound of Formula (IIb), wherein R₇ is —C(O)NR₁₀R₁₁, and R₁₀ and R₁₁ are independently substituted or unsubstituted C₁-C₈alkyl. In some embodiments is a compound of Formula (IIb), wherein R₇ is —C(O)NR₁₀R₁₁, and R₁₀ and R₁₁ are each CH₃. In some embodiments is a compound of Formula (IIb), wherein R₇ is —C(O)NR₁₀R₁₁, R₁₀ is hydrogen, and R₁ is substituted or unsubstituted C₁-C₈alkyl. In some embodiments is a compound of Formula (IIb), wherein R₇ is —C(O)NR₁₀R₁₁, R₁₀ is hydrogen, and R₁ is —CH₃. In some embodiments is a compound of Formula (IIb), wherein R₇ is —C(O)NR₁₀R₁₁, R₁₀ is substituted or unsubstituted aryl, and R₁ is substituted or unsubstituted C₁-C₈alkyl. In some embodiments is a compound of Formula (IIb), wherein R₇ is —C(O)NR₁₀R₁₁, R₁₀ is substituted or unsubstituted aryl, and R₁ is hydrogen.

In some embodiments of any one of the aspects described herein, the compound of Formula (I) is of Formula (III):

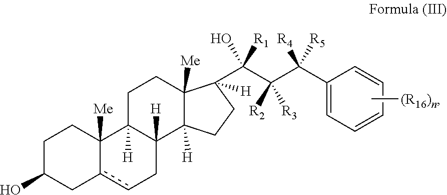

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, 2 or 3.

In some embodiments is a compound of Formula (III) wherein [symbol] is a single bond. In some embodiments is a compound of Formula (III) wherein [symbol] is a double bond.

In some embodiments is a compound of Formula (III), wherein n is 0. In some embodiments is a compound of Formula (III), wherein n is 1 and R₁₆ is halogen. In some embodiments is a compound of Formula (III), wherein n is 1 and R₁₆ is F. In some embodiments is a compound of Formula (III), wherein n is 1 and R₁₆ is Cl. In some embodiments is a compound of Formula (III), wherein n is 1 and R₁₆ is Br. In some embodiments is a compound of Formula (III), wherein n is 1 and R₁₆ is hydroxy. In some embodiments is a compound of Formula (III), wherein n is 1 and R₁₆ is substituted or unsubstituted C₁-C₈alkyl. In some embodiments is a compound of Formula (III), wherein n is 1 and R₁₆ is unsubstituted C₁-C₈alkyl. In some embodiments is a compound of Formula (III), wherein n is 1 and R₁₆ is substituted or unsubstituted C₁-C₈alkoxy. In some embodiments is a compound of Formula (III), wherein n is 2 and each R₁₆ is halogen. In some embodiments is a compound of Formula (III), wherein n is 2 and each R₁₆ is F. In some embodiments is a compound of Formula (III), wherein n is 2 and each R₁₆ is Cl. In some embodiments is a compound of Formula (III), wherein n is 2 and each R₁₆ is Br. In some embodiments is a compound of Formula (III), wherein n is 2 and one R₁₆ is halogen and one R₁₆ is hydroxy. In some embodiments is a compound of Formula (III), wherein n is 2 and each R₁₆ is hydroxy. In some embodiments is a compound of Formula (III), wherein n is 2 and one R₁₆ is halogen and one R₁₆ is substituted or unsubstituted C₁-C₈alkyl. In some embodiments is a compound of Formula (III), wherein n is 2 and each R₁₆ is substituted or unsubstituted C₁-C₈alkyl. In some embodiments is a compound of Formula (III), wherein n is 2 and each R₁₆ is unsubstituted C₁-C₈alkyl. In some embodiments is a compound of Formula (III), wherein n is 2 and one R₁₆ is halogen and one R₁₆ is substituted or unsubstituted C₁-C₈alkoxy. In some embodiments is a compound of Formula (III), wherein n is 2 and each R₁₆ is substituted or unsubstituted C₁-C₈alkoxy.

In some embodiments is a compound of Formula (III), wherein R₂, R₃, R₄, and R₅ are each deuterium. In some embodiments is a compound of Formula (III), wherein R₂, R₃, R₄, and R₅ are each hydrogen. In some embodiments is a compound of Formula (III), wherein R₂ is —OH, and R₃, R₄, and R₅ are each hydrogen. In some embodiments is a compound of Formula (III), wherein R₃ is —OH, and R₂, R₄, and R₅ are each hydrogen. In some embodiments is a compound of Formula (III), wherein R₂ and R₄ are each —OH, and R₃ and R₅ are each hydrogen. In some embodiments is a compound of Formula (III), wherein R₂ and R₅ are each —OH, and R₃ and R₄ are each hydrogen. In some embodiments is a compound of Formula (III), wherein R₃ and R₄ are each —OH, and R₂ and R₅ are each hydrogen. In some embodiments is a compound of Formula (III), wherein R₃ and R₅ are each —OH, and R₂ and R₄ are each hydrogen.

In some embodiments is a compound of Formula (III), wherein R₁ is substituted or unsubstituted C₁-C₈alkyl. In some embodiments is a compound of Formula (III), wherein R₁ is substituted C₁-C₈alkyl. In some embodiments is a compound of Formula (III), wherein R₁ is —CF₃. In some embodiments is a compound of Formula (III), wherein R₁ is unsubstituted C₁-C₈alkyl. In some embodiments is a compound of Formula (III), wherein R₁ is —CH₃. In some embodiments is a compound of Formula (III), wherein R₁ is —CH₂CH₃. In some embodiments is a compound of Formula (III), wherein R₁ is substituted or unsubstituted aryl. In some embodiments is a compound of Formula (III), wherein R₁ is unsubstituted phenyl.

In some embodiments of any one of the aspects described herein, the compound of Formula (I) is of Formula (IIIa):

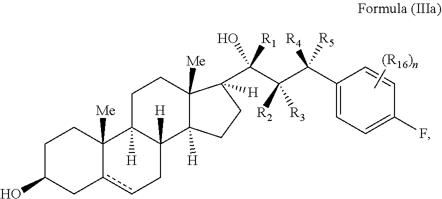

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, or 2.

In some embodiments is a compound of Formula (IIIa) wherein [symbol] is a single bond. In some embodiments is a compound of Formula (IIIa) wherein [symbol] is a double bond.

In some embodiments is a compound of Formula (IIIa), wherein n is 0. In some embodiments is a compound of Formula (IIIa), wherein n is 1 and R₁₆ is halogen. In some embodiments is a compound of Formula (IIIa), wherein n is 1 and R₁₆ is F. In some embodiments is a compound of Formula (IIIa), wherein n is 1 and R₁₆ is Cl. In some embodiments is a compound of Formula (IIIa), wherein n is 1 and R₁₆ is Br. In some embodiments is a compound of Formula (IIIa), wherein n is 1 and R₁₆ is hydroxy. In some embodiments is a compound of Formula (IIIa), wherein n is 1 and R₁₆ is substituted or unsubstituted C₁-C₈alkyl. In some embodiments is a compound of Formula (IIIa), wherein n is 1 and R₁₆ is unsubstituted C₁-C₈alkyl. In some embodiments is a compound of Formula (IIIa), wherein n is 1 and R₁₆ is substituted or unsubstituted C₁-C₈alkoxy. In some embodiments is a compound of Formula (IIIa), wherein n is 2 and each R₁₆ is halogen. In some embodiments is a compound of Formula (IIIa), wherein n is 2 and each R₁₆ is F. In some embodiments is a compound of Formula (IIIa), wherein n is 2 and each R₁₆ is Cl. In some embodiments is a compound of Formula (IIIa), wherein n is 2 and each R₁₆ is Br. In some embodiments is a compound of Formula (IIIa), wherein n is 2 and one R₁₆ is halogen and one R₁₆ is hydroxy. In some embodiments is a compound of Formula (IIIa), wherein n is 2 and each R₁₆ is hydroxy. In some embodiments is a compound of Formula (IIIa), wherein n is 2 and one R₁₆ is halogen and one R₁₆ is substituted or unsubstituted C₁-C₈alkyl. In some embodiments is a compound of Formula (IIIa), wherein n is 2 and each R₁₆ is substituted or unsubstituted C₁-C₈alkyl. In some embodiments is a compound of Formula (IIIa), wherein n is 2 and each R₁₆ is unsubstituted C₁-C₈alkyl. In some embodiments is a compound of Formula (IIIa), wherein n is 2 and one R₁₆ is halogen and one R₁₆ is substituted or unsubstituted C₁-C₈alkoxy. In some embodiments is a compound of Formula (IIIa), wherein n is 2 and each R₁₆ is substituted or unsubstituted C₁-C₈alkoxy.

In some embodiments is a compound of Formula (IIIa), wherein R₂, R₃, R₄, and R₅ are each deuterium. In some embodiments is a compound of Formula (IIIa), wherein R₂, R₃, R₄, and R₅ are each hydrogen. In some embodiments is a compound of Formula (IIIa), wherein R₂ is —OH, and R₃, R₄, and R₅ are each hydrogen. In some embodiments is a compound of Formula (IIIa), wherein R₃ is —OH, and R₂, R₄, and R₅ are each hydrogen. In some embodiments is a compound of Formula (IIIa), wherein R₂ and R₄ are each —OH, and R₃ and R₅ are each hydrogen. In some embodiments is a compound of Formula (IIIa), wherein R₂ and R₅ are each —OH, and R₃ and R₄ are each hydrogen. In some embodiments is a compound of Formula (IIIa), wherein R₃ and R₄ are each —OH, and R₂ and R₅ are each hydrogen. In some embodiments is a compound of Formula (IIIa), wherein R₃ and R₅ are each —OH, and R₂ and R₄ are each hydrogen.

In some embodiments is a compound of Formula (IIIa), wherein R₁ is substituted or unsubstituted C₁-C₈alkyl. In some embodiments is a compound of Formula (IIIa), wherein R₁ is substituted C₁-C₈alkyl. In some embodiments is a compound of Formula (IIIa), wherein R₁ is —CF₃. In some embodiments is a compound of Formula (IIIa), wherein R₁ is unsubstituted C₁-C₈alkyl. In some embodiments is a compound of Formula (IIIa), wherein R₁ is —CH₃. In some embodiments is a compound of Formula (IIIa), wherein R₁ is —CH₂CH₃. In some embodiments is a compound of Formula (IIIa), wherein R₁ is substituted or unsubstituted aryl. In some embodiments is a compound of Formula (IIIa), wherein R₁ is unsubstituted phenyl.

In some embodiments of any one of the aspects described herein, the compound of Formula (I) is of Formula (IV):

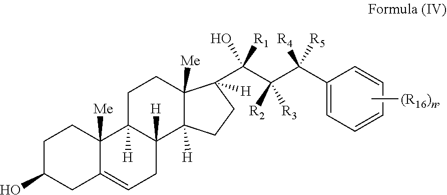

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, 2, or 3.

In some embodiments is a compound of Formula (IV), wherein n is 0. In some embodiments is a compound of Formula (IV), wherein n is 1 and R₁₆ is halogen. In some embodiments is a compound of Formula (IV), wherein n is 1 and R₁₆ is F. In some embodiments is a compound of Formula (IV), wherein n is 1 and R₁₆ is Cl. In some embodiments is a compound of Formula (IV), wherein n is 1 and R₁₆ is Br. In some embodiments is a compound of Formula (IV), wherein n is 1 and R₁₆ is hydroxy. In some embodiments is a compound of Formula (IV), wherein n is 1 and R₁₆ is substituted or unsubstituted C₁-C₈alkyl. In some embodiments is a compound of Formula (IV), wherein n is 1 and R₁₆ is unsubstituted C₁-C₈alkyl. In some embodiments is a compound of Formula (IV), wherein n is 1 and R₁₆ is substituted or unsubstituted C₁-C₈alkoxy. In some embodiments is a compound of Formula (IV), wherein n is 2 and each R₁₆ is halogen. In some embodiments is a compound of Formula (IV), wherein n is 2 and each R₁₆ is F. In some embodiments is a compound of Formula (IV), wherein n is 2 and each R₁₆ is Cl. In some embodiments is a compound of Formula (IV), wherein n is 2 and each R₁₆ is Br. In some embodiments is a compound of Formula (IV), wherein n is 2 and one R₁₆ is halogen and one R₁₆ is hydroxy. In some embodiments is a compound of Formula (IV), wherein n is 2 and each R₁₆ is hydroxy. In some embodiments is a compound of Formula (IV), wherein n is 2 and one R₁₆ is halogen and one R₁₆ is substituted or unsubstituted C₁-C₈alkyl. In some embodiments is a compound of Formula (IV), wherein n is 2 and each R₁₆ is substituted or unsubstituted C₁-C₈alkyl. In some embodiments is a compound of Formula (IV), wherein n is 2 and each R₁₆ is unsubstituted C₁-C₈alkyl. In some embodiments is a compound of Formula (IV), wherein n is 2 and one R₁₆ is halogen and one R₁₆ is substituted or unsubstituted C₁-C₈alkoxy. In some embodiments is a compound of Formula (IV), wherein n is 2 and each R₁₆ is substituted or unsubstituted C₁-C₈alkoxy.

In some embodiments is a compound of Formula (IV), wherein R₂, R₃, R₄, and R₅ are each deuterium. In some embodiments is a compound of Formula (IV), wherein R₂, R₃, R₄, and R₅ are each hydrogen. In some embodiments is a compound of Formula (IV), wherein R₂ is —OH, and R₃, R₄, and R₅ are each hydrogen. In some embodiments is a compound of Formula (IV), wherein R₃ is —OH, and R₂, R₄, and R₅ are each hydrogen. In some embodiments is a compound of Formula (IV), wherein R₂ and R₄ are each —OH, and R₃ and R₅ are each hydrogen. In some embodiments is a compound of Formula (IV), wherein R₂ and R₅ are each —OH, and R₃ and R₄ are each hydrogen. In some embodiments is a compound of Formula (IV), wherein R₃ and R₄ are each —OH, and R₂ and R₅ are each hydrogen. In some embodiments is a compound of Formula (IV), wherein R₃ and R₅ are each —OH, and R₂ and R₄ are each hydrogen.

In some embodiments is a compound of Formula (IV), wherein R₁ is substituted or unsubstituted C₁-C₈alkyl. In some embodiments is a compound of Formula (IV), wherein R₁ is substituted C₁-C₈alkyl. In some embodiments is a compound of Formula (IV), wherein R₁ is —CF₃. In some embodiments is a compound of Formula (IV), wherein R₁ is unsubstituted C₁-C₈alkyl. In some embodiments is a compound of Formula (IV), wherein R₁ is —CH₃. In some embodiments is a compound of Formula (IV), wherein R₁ is —CH₂CH₃. In some embodiments is a compound of Formula (IV), wherein R₁ is substituted or unsubstituted aryl. In some embodiments is a compound of Formula (IV), wherein R₁ is unsubstituted phenyl.

In some embodiments of any one of the aspects described herein, the compound of Formula (I) is of Formula (IVa):

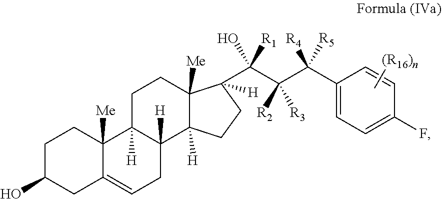

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, or 2.

In some embodiments is a compound of Formula (IVa), wherein n is 0. In some embodiments is a compound of Formula (IVa), wherein n is 1 and R₁₆ is halogen. In some embodiments is a compound of Formula (IVa), wherein n is 1 and R₁₆ is F. In some embodiments is a compound of Formula (IVa), wherein n is 1 and R₁₆ is Cl. In some embodiments is a compound of Formula (IVa), wherein n is 1 and R₁₆ is Br. In some embodiments is a compound of Formula (IVa), wherein n is 1 and R₁₆ is hydroxy. In some embodiments is a compound of Formula (IVa), wherein n is 1 and R₁₆ is substituted or unsubstituted C₁-C₈alkyl. In some embodiments is a compound of Formula (IVa), wherein n is 1 and R₁₆ is unsubstituted C₁-C₈alkyl. In some embodiments is a compound of Formula (IVa), wherein n is 1 and R₁₆ is substituted or unsubstituted C₁-C₈alkoxy. In some embodiments is a compound of Formula (IVa), wherein n is 2 and each R₁₆ is halogen. In some embodiments is a compound of Formula (IVa), wherein n is 2 and each R₁₆ is F. In some embodiments is a compound of Formula (IVa), wherein n is 2 and each R₁₆ is Cl. In some embodiments is a compound of Formula (IVa), wherein n is 2 and each R₁₆ is Br. In some embodiments is a compound of Formula (IVa), wherein n is 2 and one R₁₆ is halogen and one R₁₆ is hydroxy. In some embodiments is a compound of Formula (IVa), wherein n is 2 and each R₁₆ is hydroxy. In some embodiments is a compound of Formula (IVa), wherein n is 2 and one R₁₆ is halogen and one R₁₆ is substituted or unsubstituted C₁-C₈alkyl. In some embodiments is a compound of Formula (IVa), wherein n is 2 and each R₁₆ is substituted or unsubstituted C₁-C₈alkyl. In some embodiments is a compound of Formula (IVa), wherein n is 2 and each R₁₆ is unsubstituted C₁-C₈alkyl. In some embodiments is a compound of Formula (IVa), wherein n is 2 and one R₁₆ is halogen and one R₁₆ is substituted or unsubstituted C₁-C₈alkoxy. In some embodiments is a compound of Formula (IVa), wherein n is 2 and each R₁₆ is substituted or unsubstituted C₁-C₈alkoxy.

In some embodiments is a compound of Formula (IVa), wherein R₂, R₃, R₄, and R₅ are each deuterium. In some embodiments is a compound of Formula (IVa), wherein R₂, R₃, R₄, and R₅ are each hydrogen. In some embodiments is a compound of Formula (IVa), wherein R₂ is —OH, and R₃, R₄, and R₅ are each hydrogen. In some embodiments is a compound of Formula (IVa), wherein R₃ is —OH, and R₂, R₄, and R₅ are each hydrogen. In some embodiments is a compound of Formula (IVa), wherein R₂ and R₄ are each —OH, and R₃ and R₅ are each hydrogen. In some embodiments is a compound of Formula (IVa), wherein R₂ and R₅ are each —OH, and R₃ and R₄ are each hydrogen. In some embodiments is a compound of Formula (IVa), wherein R₃ and R₄ are each —OH, and R₂ and R₅ are each hydrogen. In some embodiments is a compound of Formula (IVa), wherein R₃ and R₅ are each —OH, and R₂ and R₄ are each hydrogen.

In some embodiments is a compound of Formula (IVa), wherein R₁ is substituted or unsubstituted C₁-C₈alkyl. In some embodiments is a compound of Formula (IVa), wherein R₁ is substituted C₁-C₈alkyl. In some embodiments is a compound of Formula (IVa), wherein R₁ is —CF₃. In some embodiments is a compound of Formula (IVa), wherein R₁ is unsubstituted C₁-C₈alkyl. In some embodiments is a compound of Formula (IVa), wherein R₁ is —CH₃. In some embodiments is a compound of Formula (IVa), wherein R₁ is —CH₂CH₃. In some embodiments is a compound of Formula (IVa), wherein R₁ is substituted or unsubstituted aryl. In some embodiments is a compound of Formula (IVa), wherein R₁ is unsubstituted phenyl.

In some embodiments of any one of the aspects described herein, the compound of Formula (I) is of Formula (V):

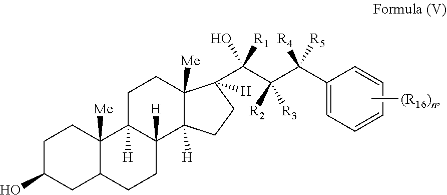

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, 2, or 3.

In some embodiments is a compound of Formula (V), wherein n is 0. In some embodiments is a compound of Formula (V), wherein n is 1 and R₁₆ is halogen. In some embodiments is a compound of Formula (V), wherein n is 1 and R₁₆ is F. In some embodiments is a compound of Formula (V), wherein n is 1 and R₁₆ is Cl. In some embodiments is a compound of Formula (V), wherein n is 1 and R₁₆ is Br. In some embodiments is a compound of Formula (V), wherein n is 1 and R₁₆ is hydroxy. In some embodiments is a compound of Formula (V), wherein n is 1 and R₁₆ is substituted or unsubstituted C₁-C₈alkyl. In some embodiments is a compound of Formula (V), wherein n is 1 and R₁₆ is unsubstituted C₁-C₈alkyl. In some embodiments is a compound of Formula (V), wherein n is 1 and R₁₆ is substituted or unsubstituted C₁-C₈alkoxy. In some embodiments is a compound of Formula (V), wherein n is 2 and each R₁₆ is halogen. In some embodiments is a compound of Formula (V), wherein n is 2 and each R₁₆ is F. In some embodiments is a compound of Formula (V), wherein n is 2 and each R₁₆ is Cl. In some embodiments is a compound of Formula (V), wherein n is 2 and each R₁₆ is Br. In some embodiments is a compound of Formula (V), wherein n is 2 and one R₁₆ is halogen and one R₁₆ is hydroxy. In some embodiments is a compound of Formula (V), wherein n is 2 and each R₁₆ is hydroxy. In some embodiments is a compound of Formula (V), wherein n is 2 and one R₁₆ is halogen and one R₁₆ is substituted or unsubstituted C₁-C₈alkyl. In some embodiments is a compound of Formula (V), wherein n is 2 and each R₁₆ is substituted or unsubstituted C₁-C₈alkyl. In some embodiments is a compound of Formula (V), wherein n is 2 and each R₁₆ is unsubstituted C₁-C₈alkyl. In some embodiments is a compound of Formula (V), wherein n is 2 and one R₁₆ is halogen and one R₁₆ is substituted or unsubstituted C₁-C₈alkoxy. In some embodiments is a compound of Formula (V), wherein n is 2 and each R₁₆ is substituted or unsubstituted C₁-C₈alkoxy.

In some embodiments is a compound of Formula (V), wherein R₂, R₃, R₄, and R₅ are each deuterium. In some embodiments is a compound of Formula (V), wherein R₂, R₃, R₄, and R₅ are each hydrogen. In some embodiments is a compound of Formula (V), wherein R₂ is —OH, and R₃, R₄, and R₅ are each hydrogen. In some embodiments is a compound of Formula (V), wherein R₃ is —OH, and R₂, R₄, and R₅ are each hydrogen. In some embodiments is a compound of Formula (V), wherein R₂ and R₄ are each —OH, and R₃ and R₅ are each hydrogen. In some embodiments is a compound of Formula (V), wherein R₂ and R₅ are each —OH, and R₃ and R₄ are each hydrogen. In some embodiments is a compound of Formula (V), wherein R₃ and R₄ are each —OH, and R₂ and R₅ are each hydrogen. In some embodiments is a compound of Formula (V), wherein R₃ and R₅ are each —OH, and R₂ and R₄ are each hydrogen.

In some embodiments is a compound of Formula (V), wherein R₁ is substituted or unsubstituted C₁-C₈alkyl. In some embodiments is a compound of Formula (V), wherein R₁ is substituted C₁-C₈alkyl. In some embodiments is a compound of Formula (V), wherein R₁ is —CF₃. In some embodiments is a compound of Formula (V), wherein R₁ is unsubstituted C₁-C₈alkyl. In some embodiments is a compound of Formula (V), wherein R₁ is —CH₃. In some embodiments is a compound of Formula (V), wherein R₁ is —CH₂CH₃. In some embodiments is a compound of Formula (V), wherein R₁ is substituted or unsubstituted aryl. In some embodiments is a compound of Formula (V), wherein R₁ is unsubstituted phenyl.

In some embodiments of any one of the aspects described herein, the compound of Formula (I) is of Formula (Va):

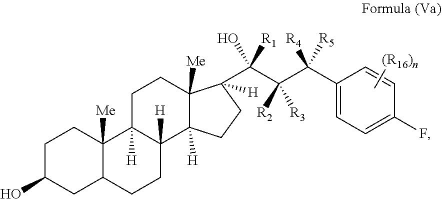

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, or 2.

In some embodiments is a compound of Formula (Va), wherein n is 0. In some embodiments is a compound of Formula (Va), wherein n is 1 and R₁₆ is halogen. In some embodiments is a compound of Formula (Va), wherein n is 1 and R₁₆ is F. In some embodiments is a compound of Formula (Va), wherein n is 1 and R₁₆ is Cl. In some embodiments is a compound of Formula (Va), wherein n is 1 and R₁₆ is Br. In some embodiments is a compound of Formula (Va), wherein n is 1 and R₁₆ is hydroxy. In some embodiments is a compound of Formula (Va), wherein n is 1 and R₁₆ is substituted or unsubstituted C₁-C₈alkyl. In some embodiments is a compound of Formula (Va), wherein n is 1 and R₁₆ is unsubstituted C₁-C₈alkyl. In some embodiments is a compound of Formula (Va), wherein n is 1 and R₁₆ is substituted or unsubstituted C₁-C₈alkoxy. In some embodiments is a compound of Formula (Va), wherein n is 2 and each R₁₆ is halogen. In some embodiments is a compound of Formula (Va), wherein n is 2 and each R₁₆ is F. In some embodiments is a compound of Formula (Va), wherein n is 2 and each R₁₆ is Cl. In some embodiments is a compound of Formula (Va), wherein n is 2 and each R₁₆ is Br. In some embodiments is a compound of Formula (Va), wherein n is 2 and one R₁₆ is halogen and one R₁₆ is hydroxy. In some embodiments is a compound of Formula (Va), wherein n is 2 and each R₁₆ is hydroxy. In some embodiments is a compound of Formula (Va), wherein n is 2 and one R₁₆ is halogen and one R₁₆ is substituted or unsubstituted C₁-C₈alkyl. In some embodiments is a compound of Formula (Va), wherein n is 2 and each R₁₆ is substituted or unsubstituted C₁-C₈alkyl. In some embodiments is a compound of Formula (Va), wherein n is 2 and each R₁₆ is unsubstituted C₁-C₈alkyl. In some embodiments is a compound of Formula (Va), wherein n is 2 and one R₁₆ is halogen and one R₁₆ is substituted or unsubstituted C₁-C₈alkoxy. In some embodiments is a compound of Formula (Va), wherein n is 2 and each R₁₆ is substituted or unsubstituted C₁-C₈alkoxy.

In some embodiments is a compound of Formula (Va), wherein R₂, R₃, R₄, and R₅ are each deuterium. In some embodiments is a compound of Formula (Va), wherein R₂, R₃, R₄, and R₅ are each hydrogen. In some embodiments is a compound of Formula (Va), wherein R₂ is —OH, and R₃, R₄, and R₅ are each hydrogen. In some embodiments is a compound of Formula (Va), wherein R₃ is —OH, and R₂, R₄, and R₅ are each hydrogen. In some embodiments is a compound of Formula (Va), wherein R₂ and R₄ are each —OH, and R₃ and R₅ are each hydrogen. In some embodiments is a compound of Formula (Va), wherein R₂ and R₅ are each —OH, and R₃ and R₄ are each hydrogen. In some embodiments is a compound of Formula (Va), wherein R₃ and R₄ are each —OH, and R₂ and R₅ are each hydrogen. In some embodiments is a compound of Formula (Va), wherein R₃ and R₅ are each —OH, and R₂ and R₄ are each hydrogen.

In some embodiments is a compound of Formula (Va), wherein R₁ is substituted or unsubstituted C₁-C₈alkyl. In some embodiments is a compound of Formula (Va), wherein R₁ is substituted C₁-C₈alkyl. In some embodiments is a compound of Formula (Va), wherein R₁ is —CF₃. In some embodiments is a compound of Formula (Va), wherein R₁ is unsubstituted C₁-C₈alkyl. In some embodiments is a compound of Formula (Va), wherein R₁ is —CH₃. In some embodiments is a compound of Formula (Va), wherein R₁ is —CH₂CH₃. In some embodiments is a compound of Formula (Va), wherein R₁ is substituted or unsubstituted aryl. In some embodiments is a compound of Formula (Va), wherein R₁ is unsubstituted phenyl.

In some embodiments of any one of the aspects described herein, the compound of Formula (I) is of Formula (VI):

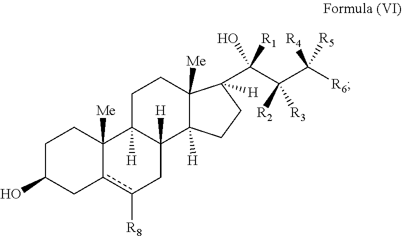

wherein: [symbol] is a single or double bond;
R₈ is hydrogen or —OH;
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VI) wherein [symbol] is a single bond. In some embodiments of any one of the aspects described herein, the compound is of Formula (VI) wherein [symbol] is a double bond.

In some embodiments of any one of the aspects described herein, the compound is of Formula (VI), wherein R₈ is hydrogen. In some embodiments of any one of the aspects described herein, the compound is of Formula (VI), wherein R₈ is —OH.

In some embodiments of any one of the aspects described herein, the compound is of Formula (VI), wherein R₆ is C₆-C₁₀aryl optionally substituted with 1, 2, 3, or 4 R₉ groups. In some embodiments of any one of the aspects described herein, the compound is of Formula (VI), wherein R₆ is C₆-C₁₀aryl substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is halogen. In some embodiments of any one of the aspects described herein, the compound is of Formula (VI), wherein R₆ is phenyl substituted with 1 R₉ group and R₉ is fluoro. In some embodiments of any one of the aspects described herein, the compound is of Formula (VI), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is phenyl substituted with 1 R₉ group and R₉ is unsubstituted C₁₋₆alkyl. In some embodiments of any one of the aspects described herein, the compound is of Formula (VI), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is unsubstituted phenyl.

In some embodiments of any one of the aspects described herein, the compound is of Formula (VI), wherein R₆ is unsubstituted phenyl.

In some embodiments of any one of the aspects described herein, the compound is of Formula (VI), wherein R₆ is C₂-C₉heteroaryl optionally substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is halogen. In some embodiments of any one of the aspects described herein, the compound is of Formula (VI), wherein R₆ is C₂-C₉heteroaryl substituted with 1 R₉ group and R₉ is fluoro. In some embodiments of any one of the aspects described herein, the compound is of Formula (VI), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is C₂-C₉heteroaryl substituted with 1 R₉ group and R₉ is unsubstituted C₁₋₆alkyl. In some embodiments of any one of the aspects described herein, the compound is of Formula (VI), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is unsubstituted phenyl.

In some embodiments of any one of the aspects described herein, the compound is of Formula (VI), wherein R₆ is unsubstituted C₂-C₉heteroaryl.

In some embodiments of any one of the aspects described herein, the compound is of Formula (VI), wherein R₆ is pyridyl optionally substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is halogen. In some embodiments of any one of the aspects described herein, the compound is of Formula (VI), wherein R₆ is pyridyl substituted with 1 R₉ group and R₉ is fluoro. In some embodiments of any one of the aspects described herein, the compound is of Formula (VI), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is pyridyl substituted with 1 R₉ group and R₉ is unsubstituted C₁₋₆alkyl. In some embodiments of any one of the aspects described herein, the compound is of Formula (VI), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is unsubstituted phenyl.

In some embodiments of any one of the aspects described herein, the compound is of Formula (VI), wherein R₆ is unsubstituted pyridyl.

In some embodiments of any one of the aspects described herein, the compound is of Formula (VI), wherein R₂, R₃, R₄, and R₅ are each hydrogen. In some embodiments of any one of the aspects described herein, the compound is of Formula (VI), wherein R₂, R₃, R₄, and R₅ are each deuterium. In some embodiments of any one of the aspects described herein, the compound is of Formula (VI), wherein R₂ is —OH, and R₃, R₄, and R₅ are each hydrogen. In some embodiments of any one of the aspects described herein, the compound is of Formula (VI), wherein R₃ is —OH, and R₂, R₄, and R₅ are each hydrogen. In some embodiments of any one of the aspects described herein, the compound is of Formula (VI), wherein R₂ and R₄ are each —OH, and R₃ and R₅ are each hydrogen. In some embodiments of any one of the aspects described herein, the compound is of Formula (VI), wherein R₂ and R₅ are each —OH, and R₃ and R₄ are each hydrogen. In some embodiments of any one of the aspects described herein, the compound is of Formula (VI), wherein R₃ and R₄ are each —OH, and R₂ and R₅ are each hydrogen. In some embodiments of any one of the aspects described herein, the compound is of Formula (VI), wherein R₃ and R₅ are each —OH, and R₂ and R₄ are each hydrogen.

In some embodiments of any one of the aspects described herein, the compound of Formula (I) is of Formula (VIa):

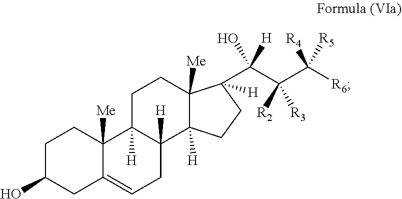

wherein:
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIa), wherein R₆ is C₆-C₁₀aryl optionally substituted with 1, 2, 3, or 4 R₉ groups. In some embodiments of any one of the aspects described herein, the compound is of Formula (VIa), wherein R₆ is C₆-C₁₀aryl substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is halogen. In some embodiments of any one of the aspects described herein, the compound is of Formula (VIa), wherein R₆ is phenyl substituted with 1 R₉ group and R₉ is fluoro. In some embodiments of any one of the aspects described herein, the compound is of Formula (VIa), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is phenyl substituted with 1 R₉ group and R₉ is unsubstituted C₁₋₆alkyl. In some embodiments of any one of the aspects described herein, the compound is of Formula (VIa), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is unsubstituted phenyl.

In some embodiments of any one of the aspects described herein, the compound is of Formula (VIa), wherein R₆ is unsubstituted phenyl.

In some embodiments of any one of the aspects described herein, the compound is of Formula (VIa), wherein R₆ is C₂-C₉heteroaryl optionally substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is halogen. In some embodiments of any one of the aspects described herein, the compound is of Formula (VIa), wherein R₆ is C₂-C₉heteroaryl substituted with 1 R₉ group and R₉ is fluoro. In some embodiments of any one of the aspects described herein, the compound is of Formula (VIa), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is C₂-C₉heteroaryl substituted with 1 R₉ group and R₉ is unsubstituted C₁₋₆alkyl. In some embodiments of any one of the aspects described herein, the compound is of Formula (VIa), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is unsubstituted phenyl.

In some embodiments of any one of the aspects described herein, the compound is of Formula (VIa), wherein R₆ is unsubstituted C₂-C₉heteroaryl.

In some embodiments of any one of the aspects described herein, the compound is of Formula (VIa), wherein R₆ is pyridyl optionally substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is halogen. In some embodiments of any one of the aspects described herein, the compound is of Formula (VIa), wherein R₆ is pyridyl substituted with 1 R₉ group and R₉ is fluoro. In some embodiments of any one of the aspects described herein, the compound is of Formula (VIa), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is pyridyl substituted with 1 R₉ group and R₉ is unsubstituted C₁₋₆alkyl. In some embodiments of any one of the aspects described herein, the compound is of Formula (VIa), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is unsubstituted phenyl.

In some embodiments of any one of the aspects described herein, the compound is of Formula (VIa), wherein R₆ is unsubstituted pyridyl.

In some embodiments of any one of the aspects described herein, the compound is of Formula (VIa), wherein R₂, R₃, R₄, and R₅ are each hydrogen. In some embodiments of any one of the aspects described herein, the compound is of Formula (VIa), wherein R₂, R₃, R₄, and R₅ are each deuterium. In some embodiments of any one of the aspects described herein, the compound is of Formula (VIa), wherein R₂ is —OH, and R₃, R₄, and R₅ are each hydrogen. In some embodiments of any one of the aspects described herein, the compound is of Formula (VIa), wherein R₃ is —OH, and R₂, R₄, and R₅ are each hydrogen. In some embodiments of any one of the aspects described herein, the compound is of Formula (VIa), wherein R₂ and R₄ are each —OH, and R₃ and R₅ are each hydrogen. In some embodiments of any one of the aspects described herein, the compound is of Formula (VIa), wherein R₂ and R₅ are each —OH, and R₃ and R₄ are each hydrogen. In some embodiments of any one of the aspects described herein, the compound is of Formula (VIa), wherein R₃ and R₄ are each —OH, and R₂ and R₅ are each hydrogen. In some embodiments of any one of the aspects described herein, the compound is of Formula (VIa), wherein R₃ and R₅ are each —OH, and R₂ and R₄ are each hydrogen.

In some embodiments of any one of the aspects described herein, the compound of Formula (I) is of Formula (VIb):

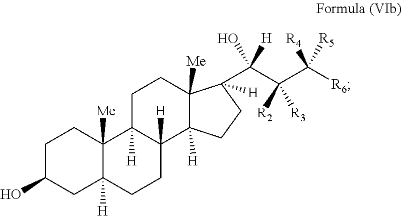

wherein:
R₂, R₃, R₄, and R₈ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIb), wherein R₆ is C₆-C₁₀aryl optionally substituted with 1, 2, 3, or 4 R₉ groups. In some embodiments of any one of the aspects described herein, the compound is of Formula (VIb), wherein R₆ is C₆-C₁₀aryl substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is halogen. In some embodiments of any one of the aspects described herein, the compound is of Formula (VIb), wherein R₆ is phenyl substituted with 1 R₉ group and R₉ is fluoro. In some embodiments of any one of the aspects described herein, the compound is of Formula (VIb), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is phenyl substituted with 1 R₉ group and R₉ is unsubstituted C₁₋₆alkyl. In some embodiments of any one of the aspects described herein, the compound is of Formula (VIb), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is unsubstituted phenyl.

In some embodiments of any one of the aspects described herein, the compound is of Formula (VIb), wherein R₆ is unsubstituted phenyl.

In some embodiments of any one of the aspects described herein, the compound is of Formula (VIb), wherein R₆ is C₂-C₉heteroaryl optionally substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is halogen. In some embodiments of any one of the aspects described herein, the compound is of Formula (VIb), wherein R₆ is C₂-C₉heteroaryl substituted with 1 R₉ group and R₉ is fluoro. In some embodiments of any one of the aspects described herein, the compound is of Formula (VIb), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is C₂-C₉heteroaryl substituted with 1 R₉ group and R₉ is unsubstituted C₁₋₆alkyl. In some embodiments of any one of the aspects described herein, the compound is of Formula (VIb), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is unsubstituted phenyl.

In some embodiments of any one of the aspects described herein, the compound is of Formula (VIb), wherein R₆ is unsubstituted C₂-C₉heteroaryl.

In some embodiments of any one of the aspects described herein, the compound is of Formula (VIb), wherein R₆ is pyridyl optionally substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is halogen. In some embodiments of any one of the aspects described herein, the compound is of Formula (VIb), wherein R₆ is pyridyl substituted with 1 R₉ group and R₉ is fluoro. In some embodiments of any one of the aspects described herein, the compound is of Formula (VIb), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is pyridyl substituted with 1 R₉ group and R₉ is unsubstituted C₁₋₆alkyl. In some embodiments of any one of the aspects described herein, the compound is of Formula (VIb), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is unsubstituted phenyl.

In some embodiments of any one of the aspects described herein, the compound is of Formula (VIb), wherein R₆ is unsubstituted pyridyl.

In some embodiments of any one of the aspects described herein, the compound is of Formula (VIb), wherein R₂, R₃, R₄, and R₅ are each hydrogen. In some embodiments of any one of the aspects described herein, the compound is of Formula (VIb), wherein R₂, R₃, R₄, and R₅ are each deuterium. In some embodiments of any one of the aspects described herein, the compound is of Formula (VIb), wherein R₂ is —OH, and R₃, R₄, and R₅ are each hydrogen. In some embodiments of any one of the aspects described herein, the compound is of Formula (VIb), wherein R₃ is —OH, and R₂, R₄, and R₅ are each hydrogen. In some embodiments of any one of the aspects described herein, the compound is of Formula (VIb), wherein R₂ and R₄ are each —OH, and R₃ and R₅ are each hydrogen. In some embodiments of any one of the aspects described herein, the compound is of Formula (VIb), wherein R₂ and R₅ are each —OH, and R₃ and R₄ are each hydrogen. In some embodiments of any one of the aspects described herein, the compound is of Formula (VIb), wherein R₃ and R₄ are each —OH, and R₂ and R₅ are each hydrogen. In some embodiments of any one of the aspects described herein, the compound is of Formula (VIb), wherein R₃ and R₅ are each —OH, and R₂ and R₄ are each hydrogen.

In some embodiments of any one of the aspects described herein, the compound of Formula (I) is of Formula (VIc):

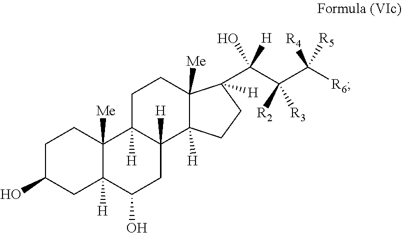

wherein:
R₂, R₃, R₄, and R₈ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIc), wherein R₆ is C₆-C₁₀aryl optionally substituted with 1, 2, 3, or 4 R₉ groups. In some embodiments of any one of the aspects described herein, the compound is of Formula (VIc), wherein R₆ is C₆-C₁₀aryl substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is halogen. In some embodiments of any one of the aspects described herein, the compound is of Formula (VIc), wherein R₆ is phenyl substituted with 1 R₉ group and R₉ is fluoro. In some embodiments of any one of the aspects described herein, the compound is of Formula (VIc), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is phenyl substituted with 1 R₉ group and R₉ is unsubstituted C₁₋₆alkyl. In some embodiments of any one of the aspects described herein, the compound is of Formula (VIc), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is unsubstituted phenyl.

In some embodiments of any one of the aspects described herein, the compound is of Formula (VIc), wherein R₆ is unsubstituted phenyl.

In some embodiments of any one of the aspects described herein, the compound is of Formula (VIc), wherein R₆ is C₂-C₉heteroaryl optionally substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is halogen. In some embodiments of any one of the aspects described herein, the compound is of Formula (VIc), wherein R₆ is C₂-C₉heteroaryl substituted with 1 R₉ group and R₉ is fluoro. In some embodiments of any one of the aspects described herein, the compound is of Formula (VIc), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is C₂-C₉heteroaryl substituted with 1 R₉ group and R₉ is unsubstituted C₁₋₆alkyl. In some embodiments of any one of the aspects described herein, the compound is of Formula (VIc), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is unsubstituted phenyl.

In some embodiments of any one of the aspects described herein, the compound is of Formula (VIc), wherein R₆ is unsubstituted C₂-C₉heteroaryl.

In some embodiments of any one of the aspects described herein, the compound is of Formula (VIc), wherein R₆ is pyridyl optionally substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is halogen. In some embodiments of any one of the aspects described herein, the compound is of Formula (VIc), wherein R₆ is pyridyl substituted with 1 R₉ group and R₉ is fluoro. In some embodiments of any one of the aspects described herein, the compound is of Formula (VIc), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is pyridyl substituted with 1 R₉ group and R₉ is unsubstituted C₁₋₆alkyl. In some embodiments of any one of the aspects described herein, the compound is of Formula (VIc), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is unsubstituted phenyl.

In some embodiments of any one of the aspects described herein, the compound is of Formula (VIc), wherein R₆ is unsubstituted pyridyl.

In some embodiments of any one of the aspects described herein, the compound is of Formula (VIc), wherein R₂, R₃, R₄, and R₅ are each hydrogen. In some embodiments of any one of the aspects described herein, the compound is of Formula (VIc), wherein R₂, R₃, R₄, and R₅ are each deuterium. In some embodiments of any one of the aspects described herein, the compound is of Formula (VIc), wherein R₂ is —OH, and R₃, R₄, and R₅ are each hydrogen. In some embodiments of any one of the aspects described herein, the compound is of Formula (VIc), wherein R₃ is —OH, and R₂, R₄, and R₅ are each hydrogen. In some embodiments of any one of the aspects described herein, the compound is of Formula (VIc), wherein R₂ and R₄ are each —OH, and R₃ and R₅ are each hydrogen. In some embodiments of any one of the aspects described herein, the compound is of Formula (VIc), wherein R₂ and R₅ are each —OH, and R₃ and R₄ are each hydrogen. In some embodiments of any one of the aspects described herein, the compound is of Formula (VIc), wherein R₃ and R₄ are each —OH, and R₂ and R₅ are each hydrogen. In some embodiments of any one of the aspects described herein, the compound is of Formula (VIc), wherein R₃ and R₅ are each —OH, and R₂ and R₄ are each hydrogen.

In some embodiments of any one of the aspects described herein, the compound of Formula (I) is of Formula (VII):

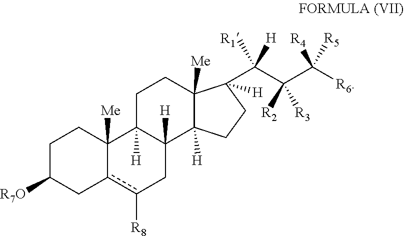

In some embodiments of any one of the aspects described herein, the compound of Formula (I) is of Formula (VIIa):

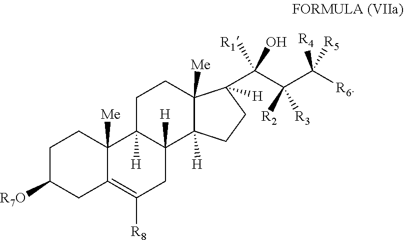

In some embodiments of any one of the aspects described herein, the compound of Formula (I) is of Formula (VIIb):

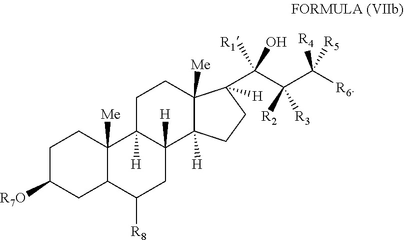

In some embodiments of the various aspect described herein, the compound is selected from the following:

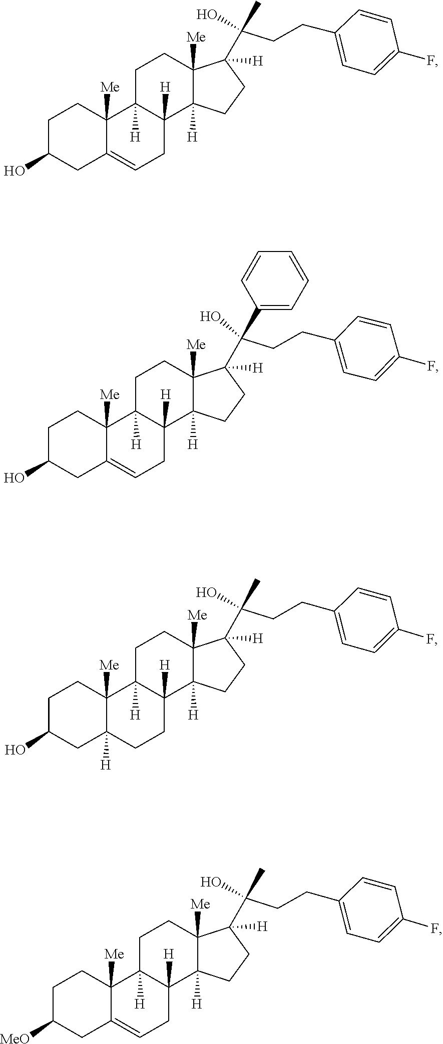

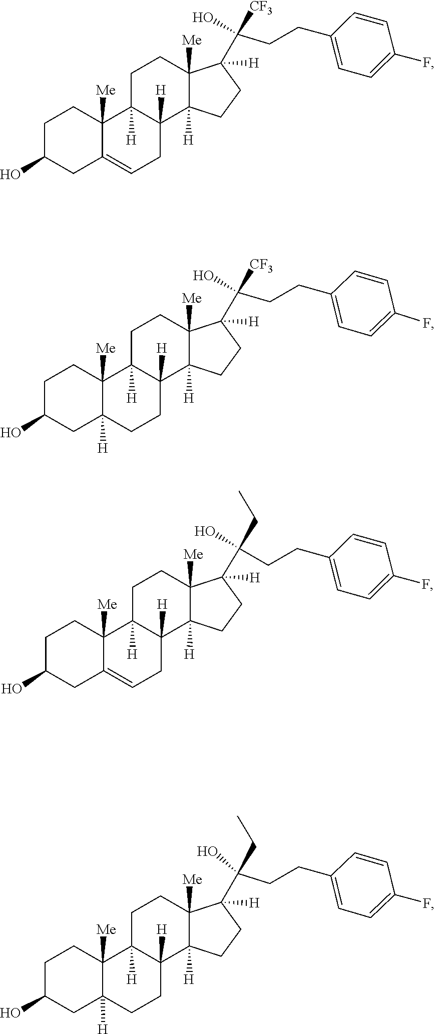

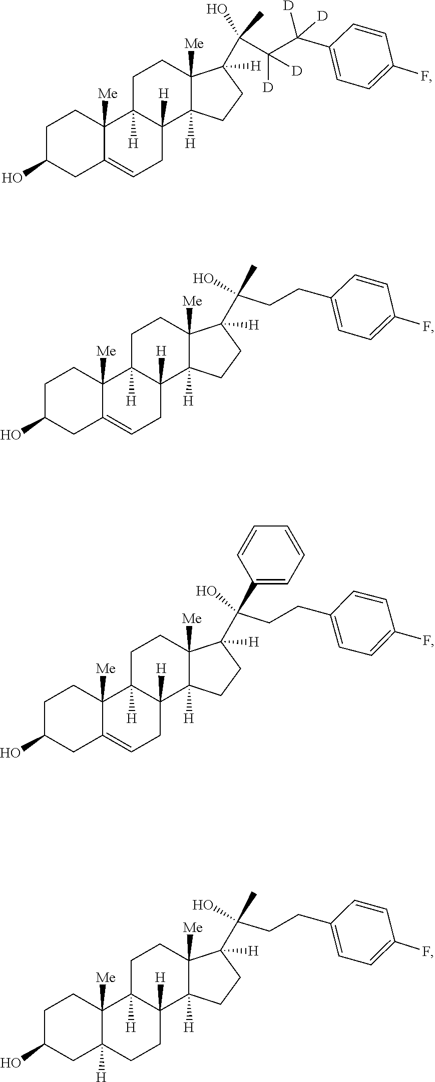

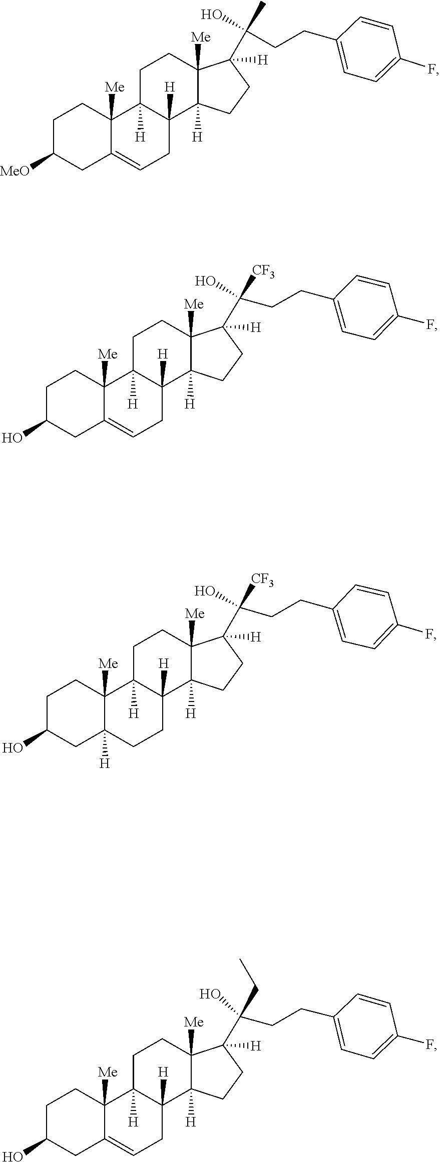

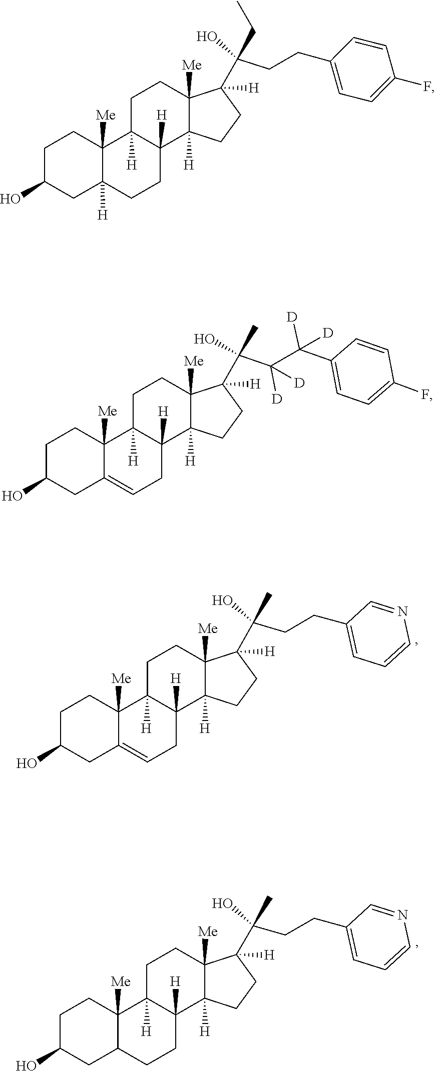

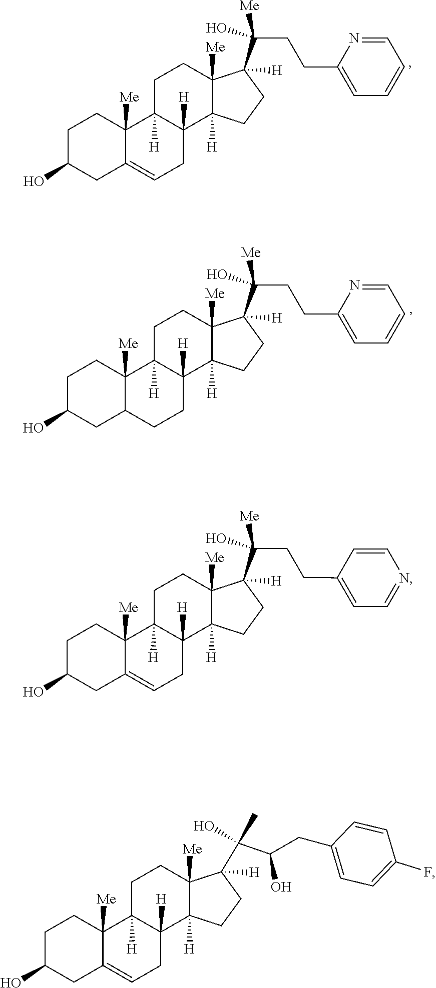

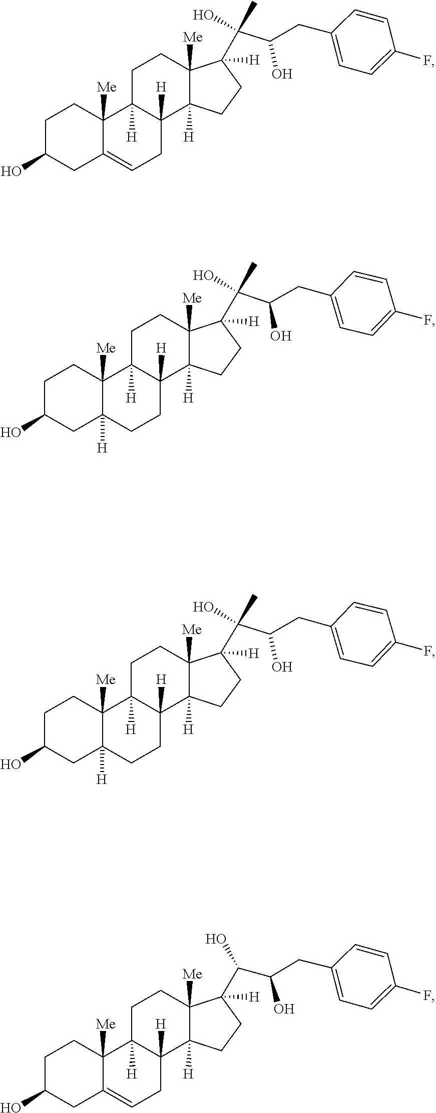

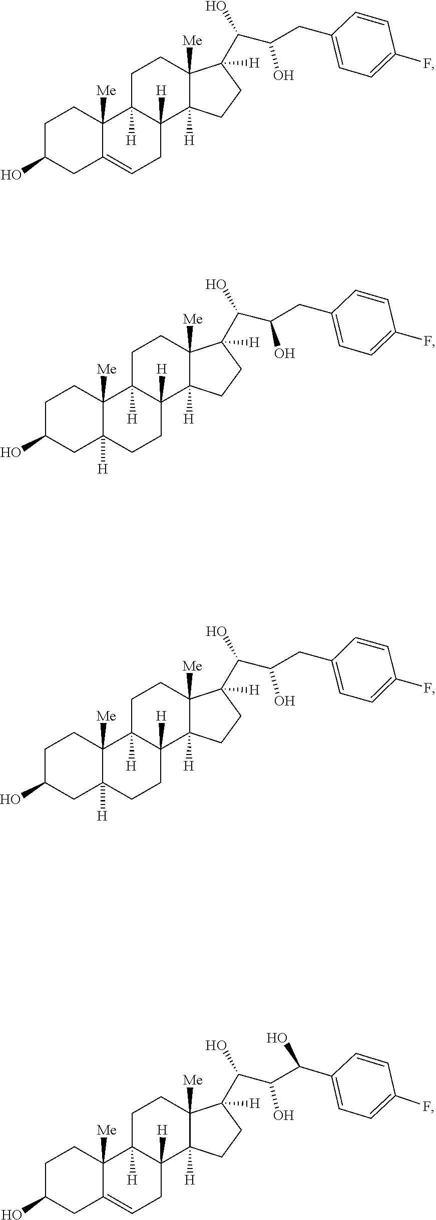

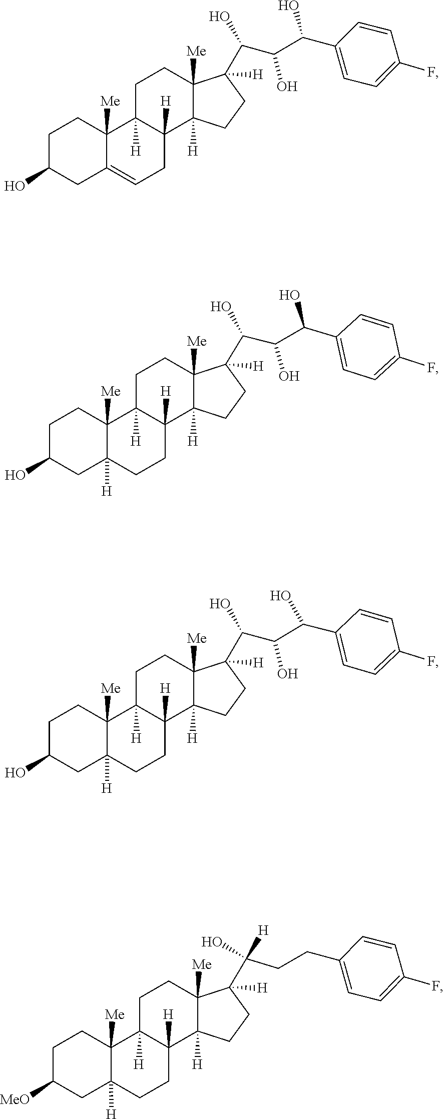

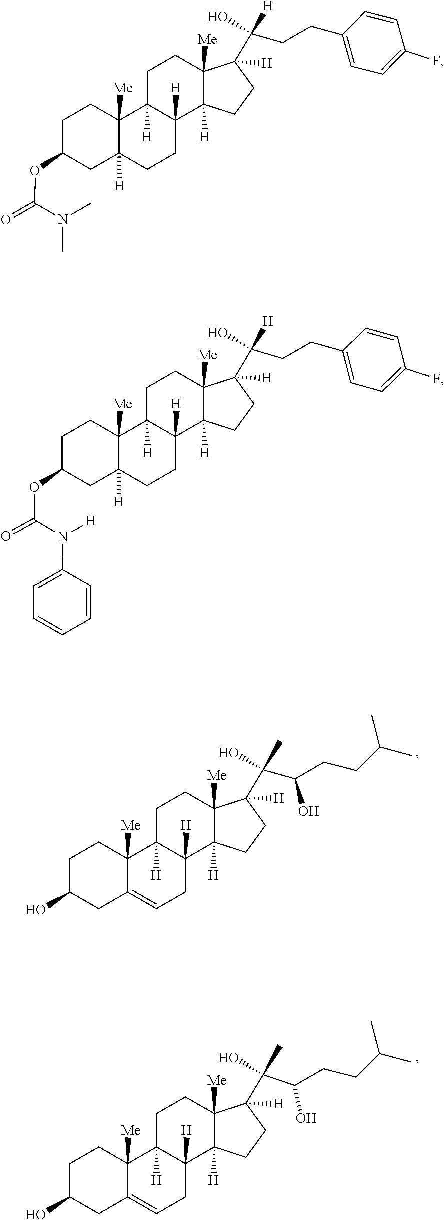

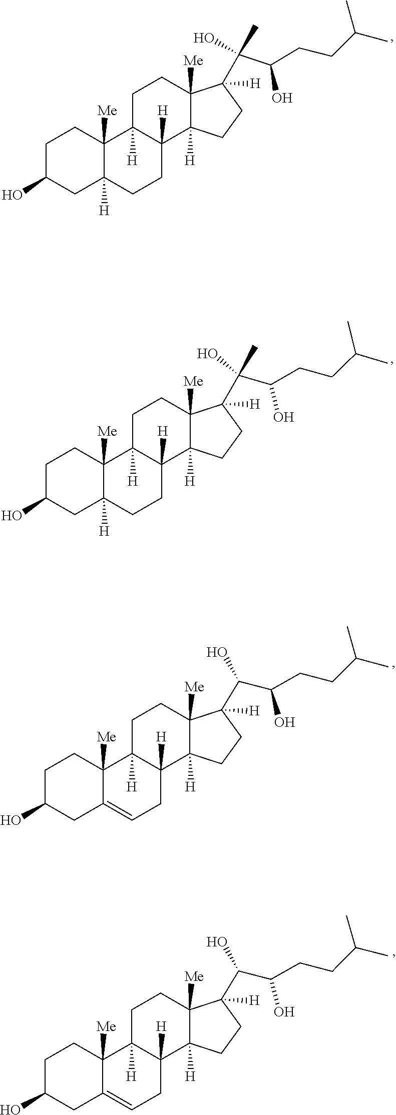

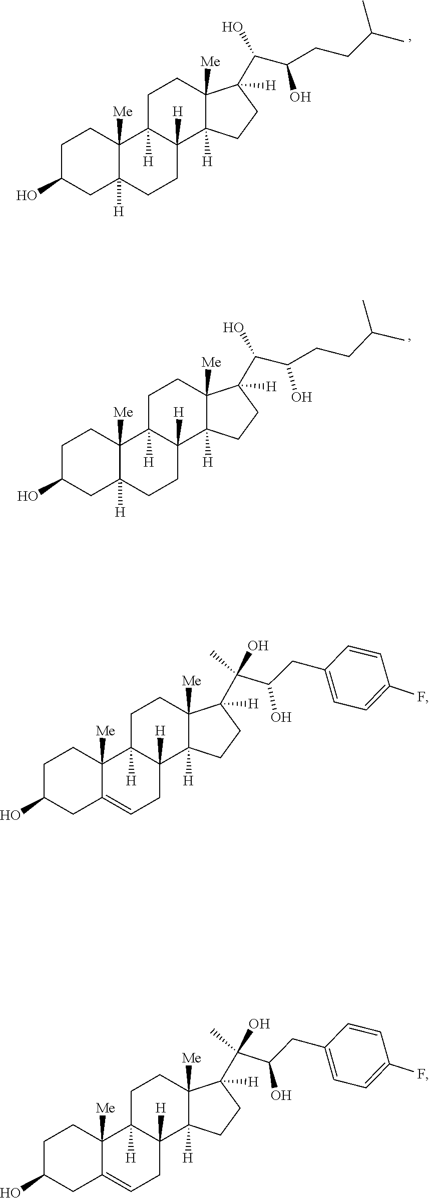

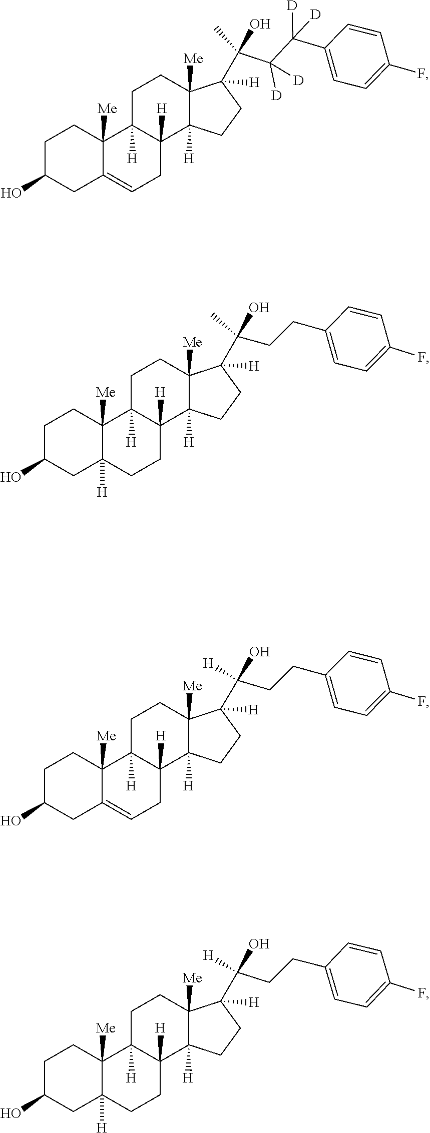

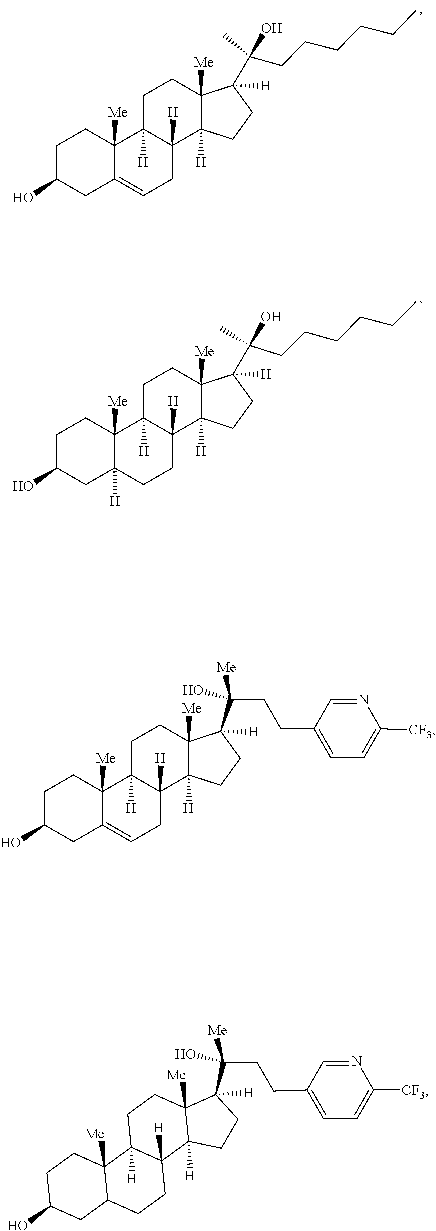

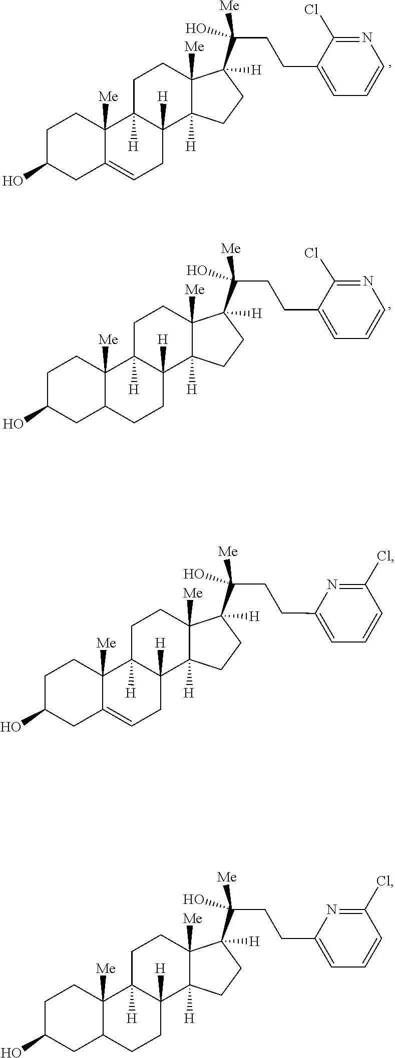

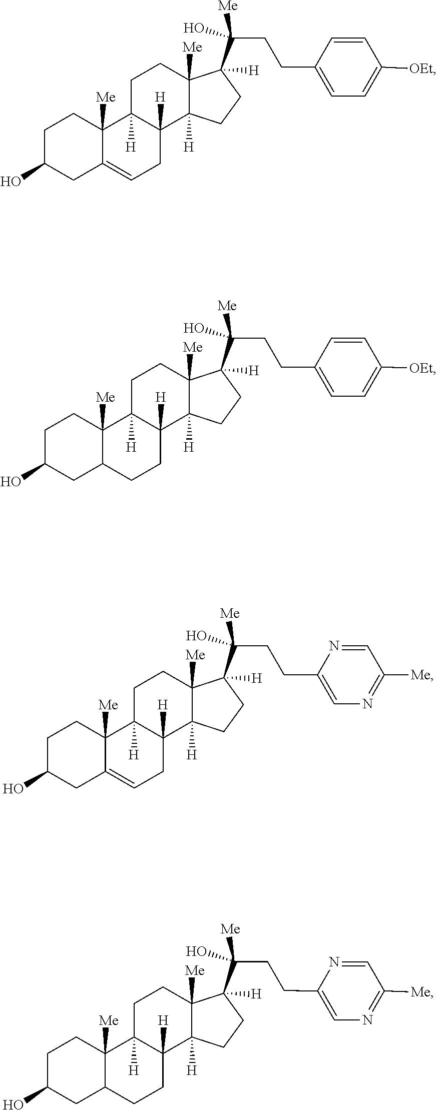

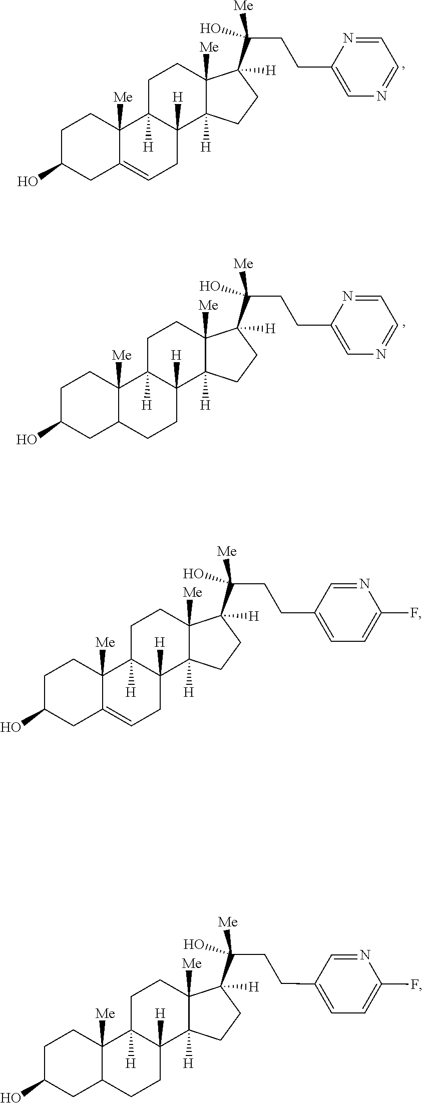

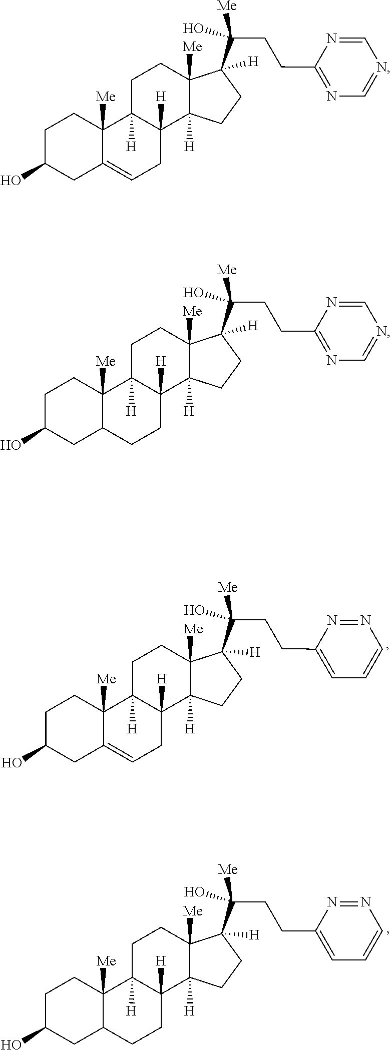

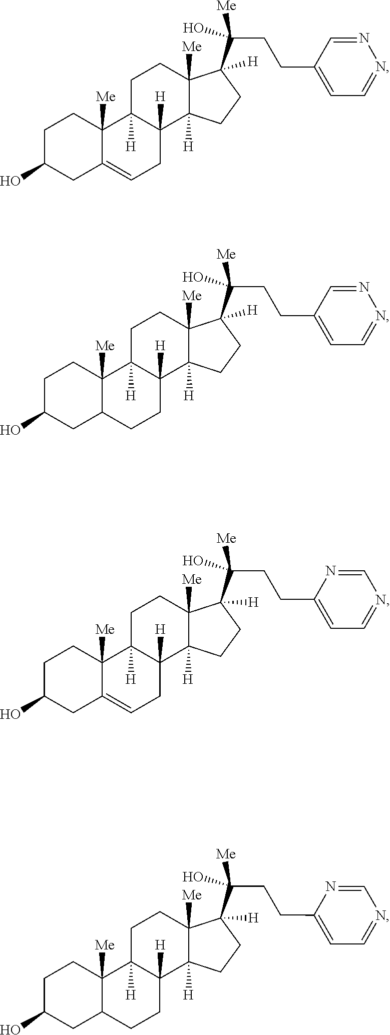

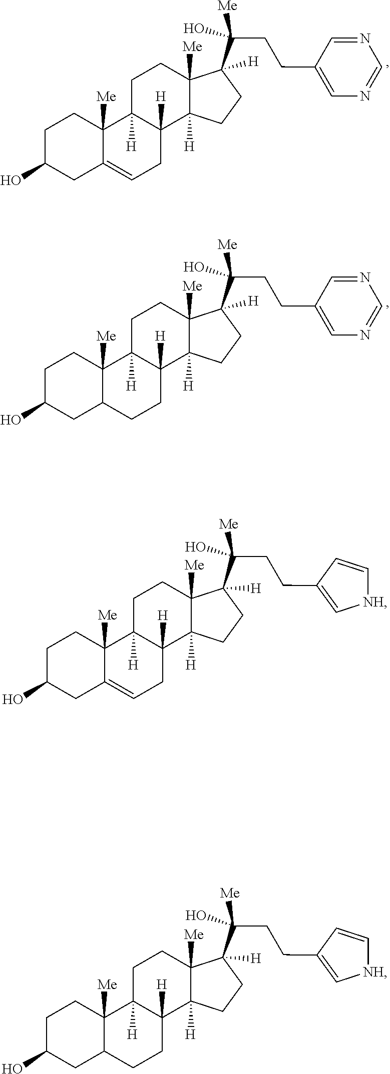

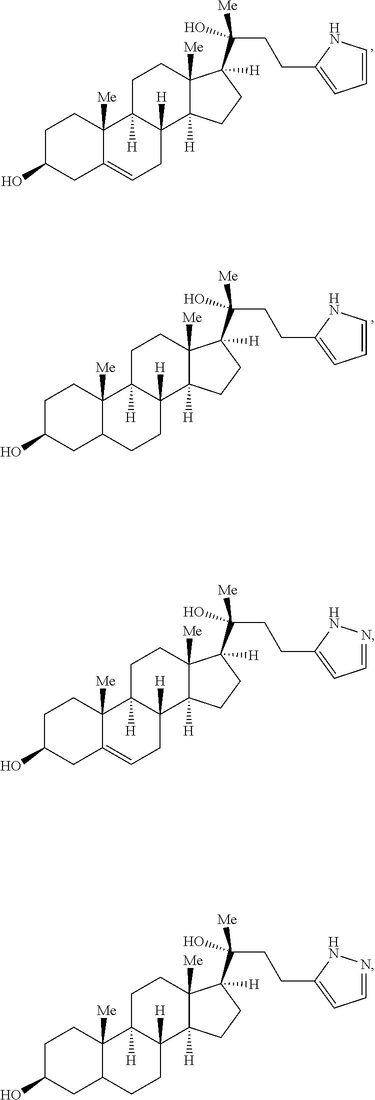

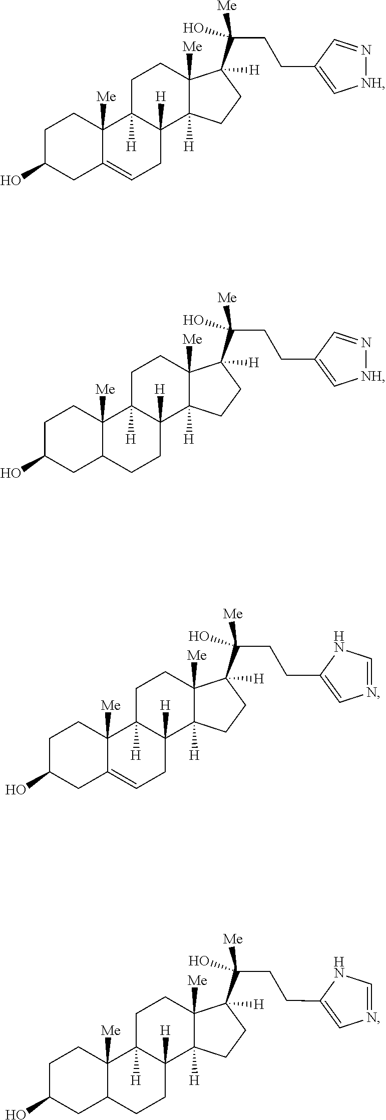

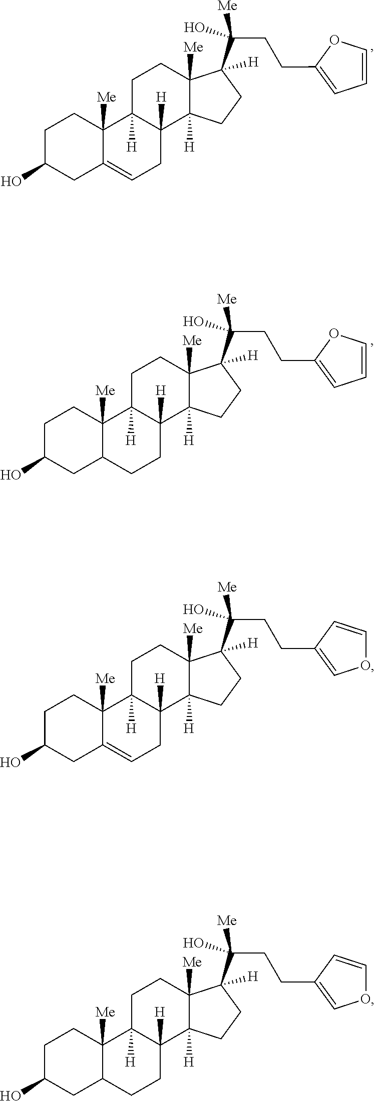

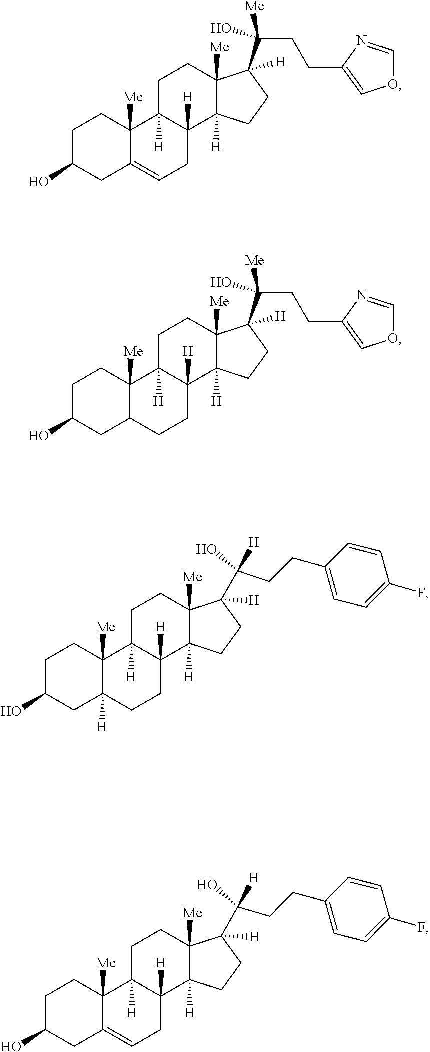

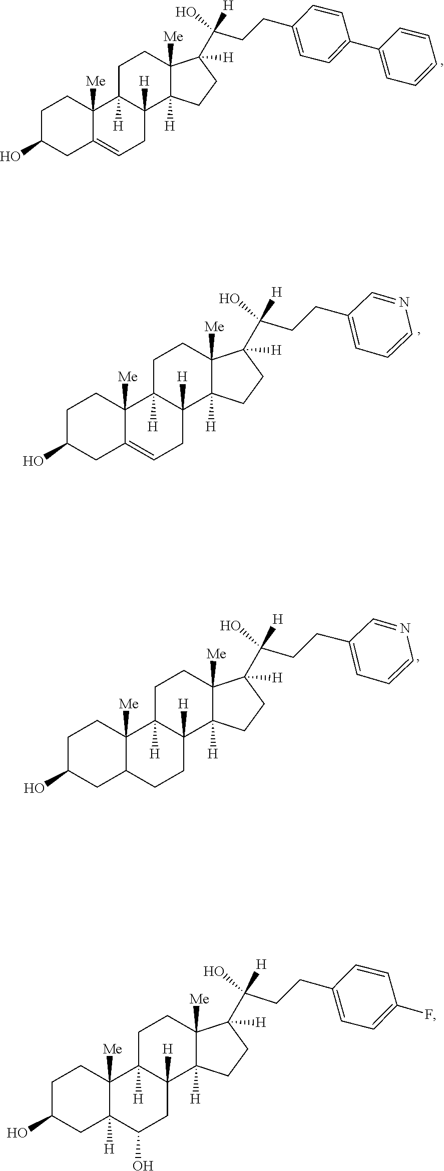

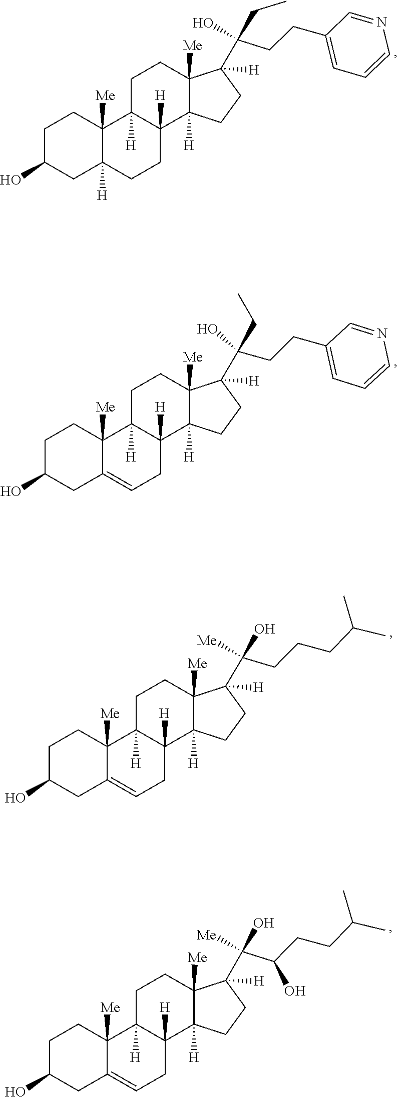

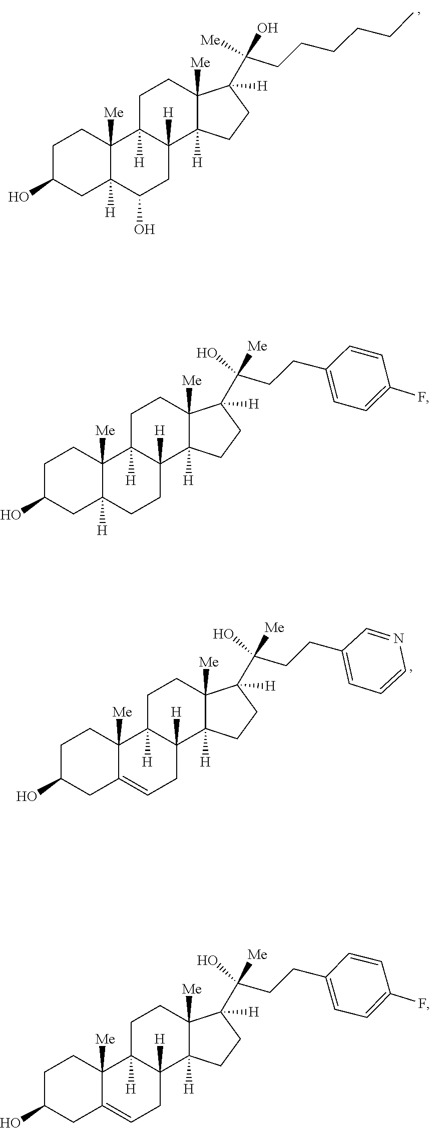

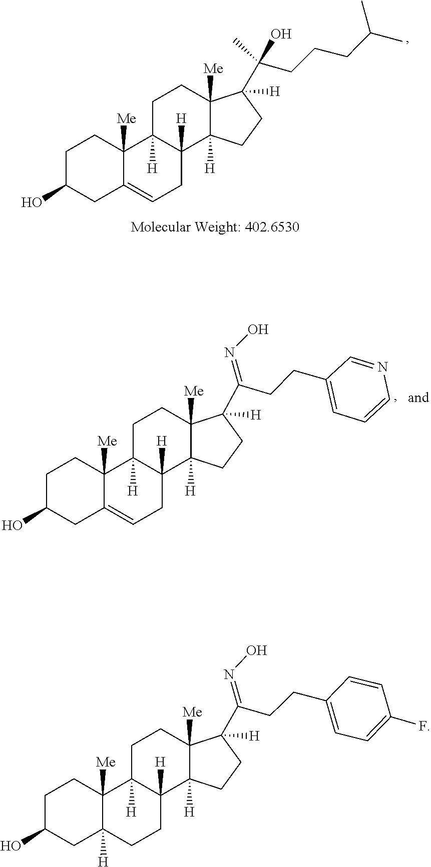

Additional exemplary compounds of Formula (I) are described, for example, in U.S. Pat. No. 9,637,514, US Patent Publication No. 20170189429, US Patent Publication No. 20180311259, and U.S. Provisional Application No. 63/035,597, filed Jun. 5, 2020, contents of all of which are incorporated herein by reference in their entireties.

### Synthesis of the Compounds

The synthesis of compounds described herein can be accomplished using means described in the chemical literature. For example, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. For example, compounds described herein can be synthesized using methods described, for example, in U.S. Pat. No. 9,637,514, US Patent Publication No. 20170189429, US Patent Publication No. 20180311259, and U.S. Provisional Application No. 63/035,597, filed Jun. 5, 2020.

### Routes of Administration

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be systemic. In some embodiments, administration can be local.

In some embodiments, a compound of Formula (I) is orally administered. Without limitations, oral administration can be in the form of solutions, suspensions, tablets, pills, capsules, sustained-release formulations, oral rinses, powders and the like.

In some embodiments, a compound of Formula (I) is compound is administered in a local rather than systemic manner, for example, via topical application of the compound directly on to skin, or intravenously, or subcutaneously, often in a depot preparation or sustained release formulation. In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

The phrase “therapeutically-effective amount” as used herein means that amount of a compound, material, or composition comprising a compound described herein which is effective for producing some desired therapeutic effect in at least a sub-population of cells, e.g., modulate or inhibit activity of MAGL in a subject at a reasonable benefit/risk ratio applicable to any medical treatment. Thus, “therapeutically effective amount” means that amount which, when administered to a subject for treating a disease, is sufficient to affect such treatment for the disease.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for a compound as disclosed herein can be about 0.01 μM to about 10 μM, about 0.2 μM to about 5 μM, or about 0.8 to about 3 μM in a subject, such as a rat, dog, or human.

Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The compounds described herein can be administered at once, or can be divided into a number of smaller doses to be administered at intervals of time. Thus, in some embodiments, the compound is administered once a day. In some other embodiments, the compound is administered multiple times, e.g., two, three, four, five or more times a day.

It is understood that the precise dosage and duration of treatment will be a function of the location of where the composition is parenterally administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The compound can be administered as a single bolus or multiple boluses, as a continuous infusion, or a combination thereof. For example, the compound can be administered as a single bolus initially, and then administered as a continuous infusion following the bolus. The rate of the infusion can be any rate sufficient to maintain effective concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

### Pharmaceutical Compositions/Formulations

For administration to a subject, the compounds of Formula (I) can be provided in pharmaceutically acceptable compositions. These pharmaceutically acceptable compositions comprise a compound of Formula (I), formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Examples of solid carriers include starch, sugar, bentonite, silica, and other commonly used carriers. Further non-limiting examples of carriers and diluents which can be used in the formulations comprising a compound of Formula (I) as disclosed herein of the present invention include saline, syrup, dextrose, and water.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. According, a “therapeutically effective amount” refers to an amount effective, at dosage and periods of time necessary, to achieve a desired therapeutic result. A therapeutic result can be, e.g., lessening of symptoms, prolonged survival, improved mobility, and the like. A therapeutic result need not be a “cure.”

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectable solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, compounds can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; content of each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like. The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound. Examples of bases useful for formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives. The compositions can be given as a bolus dose, to maximize the circulating levels for the greatest length of time after the dose. Continuous infusion may also be used after the bolus dose.

The compounds can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions

The compounds can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

For oral or enteral formulations as disclosed herein for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982. As regards formulations for administering a compound of Formula I as disclosed herein, one particularly useful embodiment

Also provided herein is a tablet formulation comprising a compound of Formula I with an enteric polymer casing. An example of such a preparation can be found in WO2005/021002. The active material in the core can be present in a micronised or solubilised form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropylcellulose, polyvinylpyrrolidone, pre-gelatinised starch or gum acacia or mixtures thereof; disintegrants such as microcrystalline cellulose (fulfilling both binder and disintegrant functions) cross-linked polyvinylpyrrolidone, sodium starch glycollate, croscarmellose sodium or mixtures thereof; lubricants, such as magnesium stearate or stearic acid, glidants or flow aids, such as colloidal silica, talc or starch, and stabilisers such as desiccating amorphous silica, colouring agents, flavours etc. Preferably the tablet comprises lactose as diluent. When a binder is present, it is preferably hydroxypropylmethyl cellulose. Preferably, the tablet comprises magnesium stearate as lubricant. Preferably the tablet comprises croscarmellose sodium as disintegrant. Preferably, the tablet comprises microcrystalline cellulose.

The diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

The active ingredient, e.g., compound of Formula I preferably comprises between 10 and 50% of the weight of the core, more preferably between 15 and 35% of the weight of the core (calculated as free base equivalent). The core can contain any therapeutically suitable dosage level of the active ingredient, but preferably contains up to 150 mg of the active ingredient. Particularly preferably, the core contains 20, 30, 40, 50, 60, 80 or 100 mg of the active ingredient. The active ingredient can be present as is or as any pharmaceutically acceptable salt. If the active ingredient is present as a salt, the weight is adjusted such that the tablet contains the desired amount of active ingredient, calculated as free base or free acid of the salt.

The core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

The core is surrounded by a casing which comprises an enteric polymer. Examples of enteric polymers are cellulose acetate phthalate, cellulose acetate succinate, methylcellulose phthalate, ethylhydroxycellulose phthalate, polyvinylacetate pthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilised in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1, 2-, 3, 4-diepoxybutane. The casing can also include starch and/or dextrin.

In some embodiments, an enteric coating materials are the commercially available Eudragit® enteric polymers such as Eudragit® L, Eudragit® S and Eudragit® NE used alone or with a plasticiser. Such coatings are normally applied using a liquid medium, and the nature of the plasticiser depends upon whether the medium is aqueous or non-aqueous. Plasticisers for use with aqueous medium include propylene glycol, triethyl citrate, acetyl triethyl citrate or Citroflex® or Citroflex® A2. Non-aqueous plasticisers include these, and also diethyl and dibutyl phthalate and dibutyl sebacate. A preferred plasticiser is Triethyl citrate. The quantity of plasticiser included will be apparent to those skilled in the art.

The casing can also include an anti-tack agent such as talc, silica or glyceryl monostearate. Preferably the anti-tack agent is glyceryl monostearate. Typically, the casing can include around 5-25 wt % Plasticizers and up to around 50 wt % of anti-tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%.

A seal coat can also be included between the core and the enteric coating. A seal coat is a coating material which can be used to protect the enteric casing from possible chemical attack by any alkaline ingredients in the core. The seal coat can also provide a smoother surface, thereby allowing easier attachment of the enteric casing. A person skilled in the art would be aware of suitable coatings. Preferably the seal coat is made of an Opadry coating, and particularly preferably it is Opadry White OY-S-28876. Other enteric-coated preparations of this sort can be prepared by one skilled in the art, using these materials or their equivalents.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspension, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

### Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%, ±1.5%, ±2%, ±2.5%, ±3%, ±3.5%, ±4%, +4.5%, or +5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

As used herein, the terms “treat,” “treatment,” “treating,” or “amelioration” are used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment). A treatment can be administered prior to the onset of the disease, for a prophylactic or preventive action. Alternatively, or additionally, the treatment can be administered after initiation of the disease or condition, for a therapeutic action.

In some embodiments, treatment is therapeutic and does not include prophylactic treatment.

The terms “co-administration” or the like, as used herein, are meant to encompass administration of the selected therapeutic agents to a single patient and are intended to include treatment regimens in which the agents are administered by the same or different route of administration or at the same or different time. The particular combination of therapies (therapeutics or procedures) to employ in such a combination regimen will take into account compatibility of the desired therapeutics and/or procedures and the desired therapeutic effect to be achieved.

As used herein, the term “subject” refers to any living organism which can be administered compound and/or pharmaceutical compositions of the present invention. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult, child and newborn subjects, whether male or female, are intended to be covered. The term “subject” is also intended to include living organisms susceptible to conditions or disease states as generally disclosed, but not limited to, throughout this specification. Examples of subjects include humans, dogs, cats, cows, goats, and mice. The term subject is further intended to include transgenic species. The term “subject” and “individual” are used interchangeably herein, and refer to an animal, for example a human or non-human mammals/animals, to whom treatment, including prophylactic treatment, with the compounds and compositions according to the present invention, is provided. The term “non-human animals” and “non-human mammals” are used interchangeably herein and include all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc.

In some embodiments, the subject is a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of a fibrotic disease or disorder.

It is noted that a human subject can be of any age, gender, race or ethnic group, e.g., Caucasian (white), Asian, African, black, African American, African European, Hispanic, Middle eastern, etc . . . .

In addition, the methods described herein can be used to treat domesticated animals and/or pets. A subject can be male or female. A subject can be one who has been previously diagnosed with or identified as suffering from or having a fibrotic disease or disorder, but need not have already undergone treatment.

In some embodiments of any one of the aspects, the subject is human.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as —N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₅)alkyl, O(C₁-C₅)alkyl, O(C₁-C₅)haloalkyl, (C₂-C₅)alkenyl, (C₂-C₅)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or wherein any alkyl, cycloalkyl or heterocyclyl is optionally substituted with oxo; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Inhibition of Basal Expression of TGFβ Target Genes by an Orally Bioavailable Oxysterol, Oxy210, in Primary Human Liver Stellate Cells

Primary human liver stellate cells were treated in DMEM containing 1% fetal bovine serum (FBS) for 24 hours. Next, they were treated with control vehicle or Oxy210. After 48 hours, RNA was extracted and analyzed by Q-RT-PCR for the expression of the genes as indicated and normalized to GAPDH expression. Results are shown in FIG. 1.

### Example 2: Inhibition of Basal Expression of Profibrogenic Genes by Oxy210 in Human Pericytes

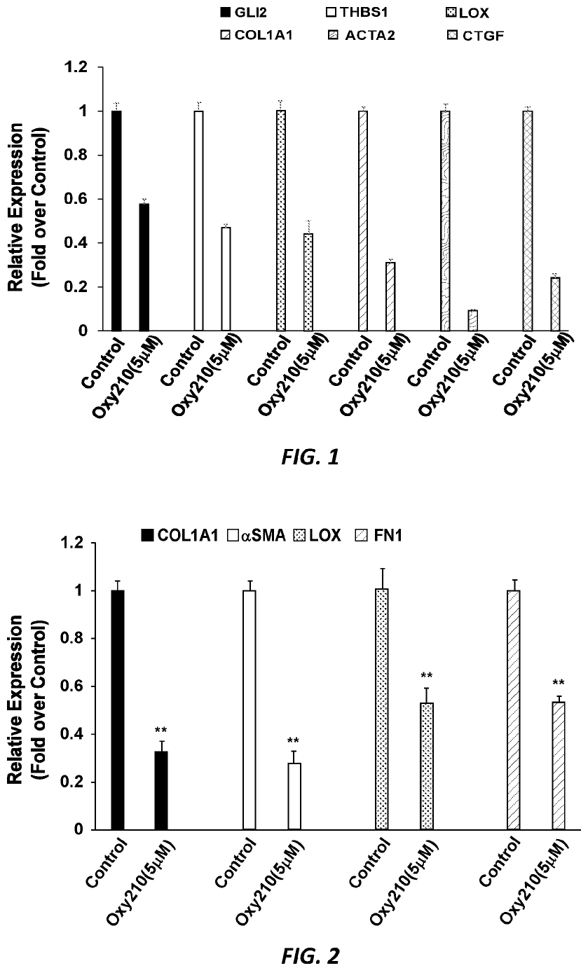

Primary human pericytes were treated with control vehicle or Oxy210 for 72 hours in DMEM containing 5% FBS. RNA was extracted and analyzed by Q-RT-PCR for the expression of profibrotic genes and normalized to GAPDH expression. Results are shown in FIG. 2.

### Example 3: Effect of Oxy210 on TGFβ-Induced Expression of Profibrotic Genes in Primary Human Pericytes

Primary human pericytes cultured in DMEM containing 1% FBS were treated with TGFβ (10 ng/mL) in the absence or presence of Oxy210 for 48 hours. RNA was extracted and analyzed by Q-RT-PCR for the expression of profibrotic genes and normalized to GAPDH expression. Results are shown in FIG. 3.

### Example 4: Inhibition of TGFβ-Induced Epithelial Mesenchymal Transition (EMT) of Renal Tubular Epithelial Cells by Oxy210

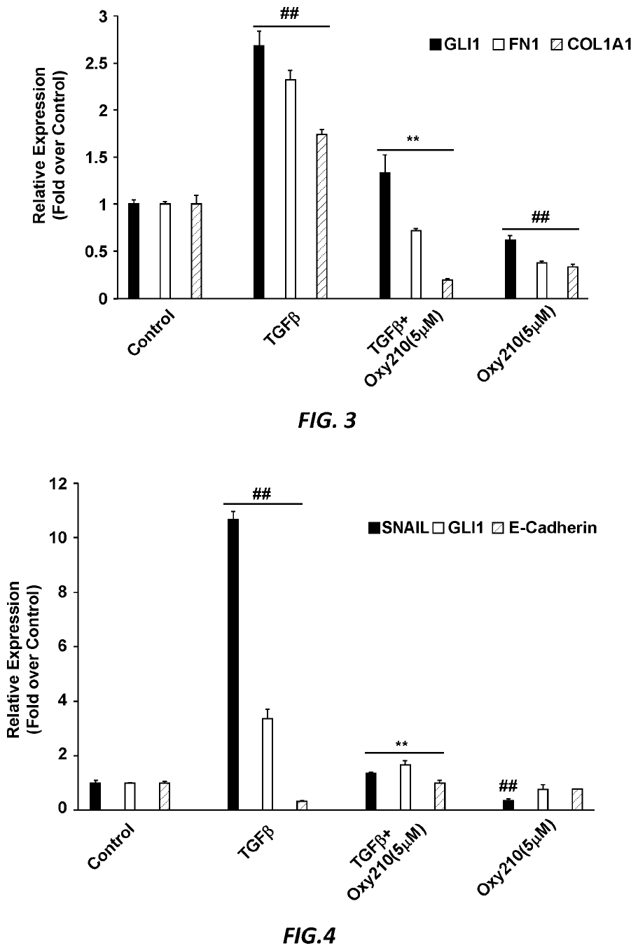

Primary human renal proximal tubular epithelial cells cultured in RPMI containing 1% FBS were treated with TGFβ (10 ng/mL) in the absence or presence of Oxy210 for 48 hours. RNA was extracted and analyzed by Q-RT-PCR for the expression of the EMT markers and Gli1 and normalized to GAPDH expression. Results are shown in FIG. 4.

### Example 5: Inhibition of Human Lung Fibroblast Cell Proliferation by Oxy210

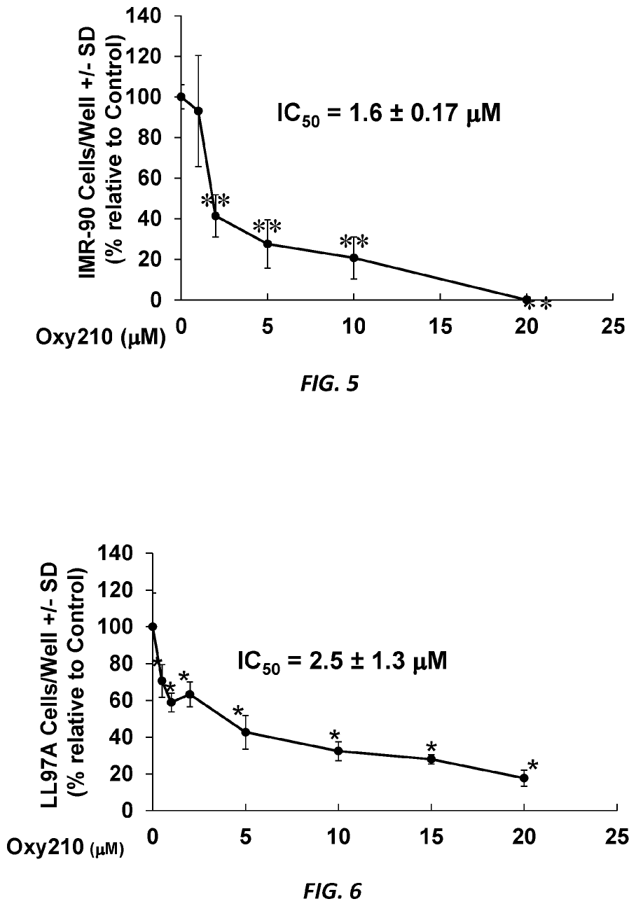

IMR-90 cells were treated with Oxy210 at the concentrations as indicated in DMEM containing 1% FBS for 5 days and then were trypsinized and counted. Results are shown in FIG. 5.

### Example 6: Inhibition of Lung Fibroblast Cell Proliferation by Oxy210

LL97A cells were treated with Oxy210 at the concentrations as indicated in F12K containing 1% FBS for 5 days and then were trypsinized and counted. Results are shown in FIG. 6.

### Example 7: Oxy210 Inhibits Basal Profibrotic Gene Expression in IMR-90 Cells

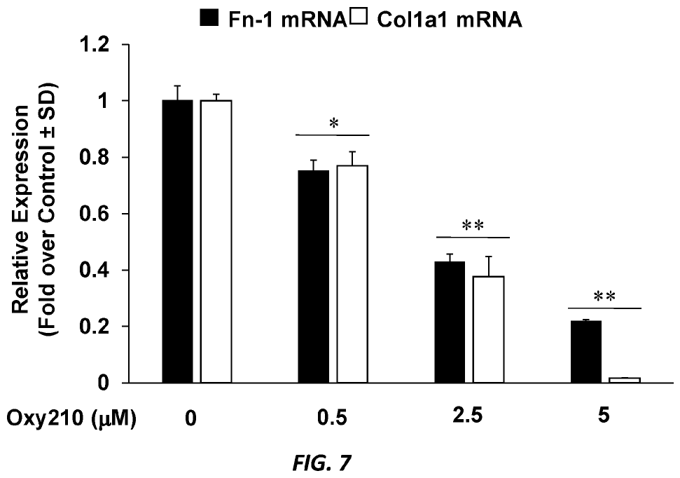

IMR-90 cells were treated in F12K medium containing 1% FBS and Oxy210 at the indicated concentrations for 72 hours. RNA was then extracted and analyzed by Q-RT-PCR for the expression of the genes as indicated and normalized to GAPDH expression. Results are shown in FIG. 7.

### Example 8: Oxy210 Inhibits TGFβ-Induced COL1A1 and a-SMA Expression in LL97A Cells

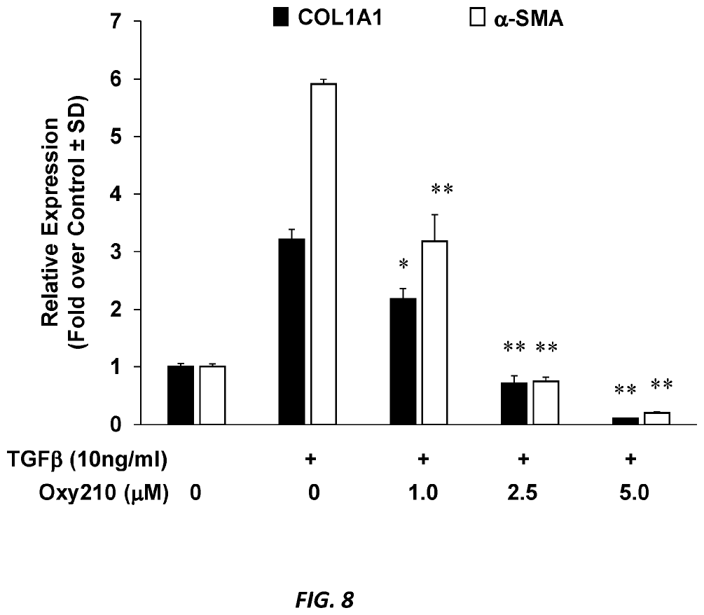

LL97A cells were pretreated in F12K medium containing 1% FBS for 24 hours and then the cells were treated with TGFβ in the absence or presence of Oxy210 at the indicated concentrations. After 48 hours, RNA was extracted and analyzed by Q-RT-PCR for the expression of the genes as indicated and normalized to GAPDH expression. Results are shown in FIG. 8.

### Example 9: Oxy210 Inhibits Basal COL1A1 Expression in LL97A Cells

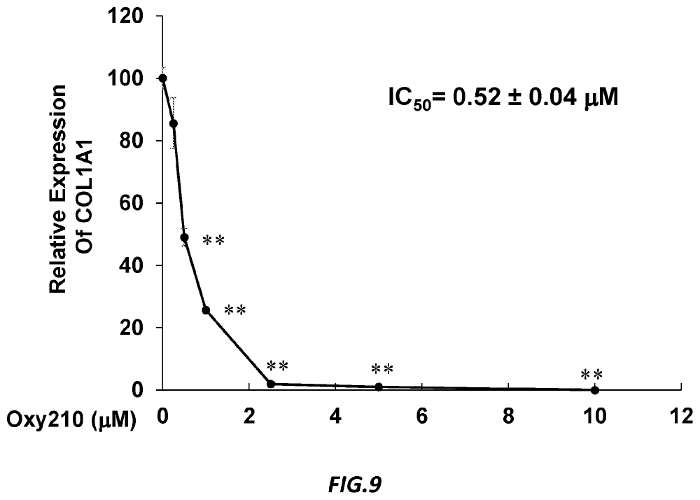

LL97A cells were treated in F12K medium containing 1% FBS and Oxy210 at the indicated concentrations for 72 hours. RNA was then extracted and analyzed by Q-RT-PCR for the expression of the genes as indicated and normalized to GAPDH expression. Results are shown in FIG. 9.

### Example 10: Effect of Oxy210 on Bleomycin-Induced Pulmonary Fibrosis in Mice

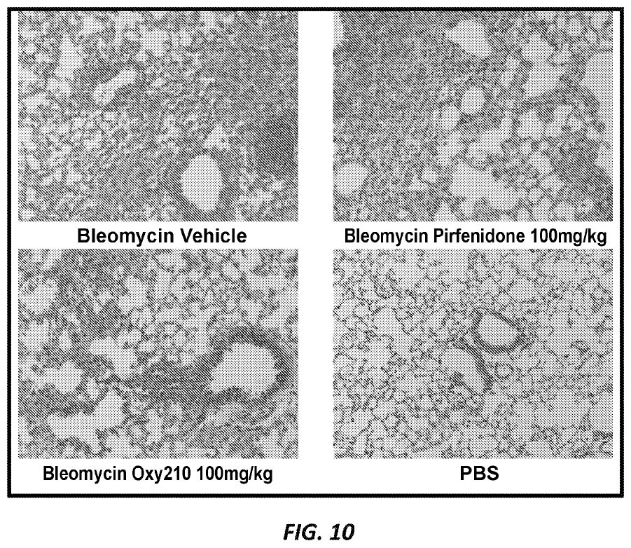

C57BL/6 mice were treated once intratracheally with bleomycin with or without pirfenidone or Oxy210. In FIG. 10, H&E staining of lung sections are shown, demonstrating a clear beneficial effect of Oxy210 on inhibiting bleomycin-induced damages to the lung architecture.

### Example 11: Effect of Oxy210 on Bleomycin-Induced Pulmonary Fibrosis in Mice

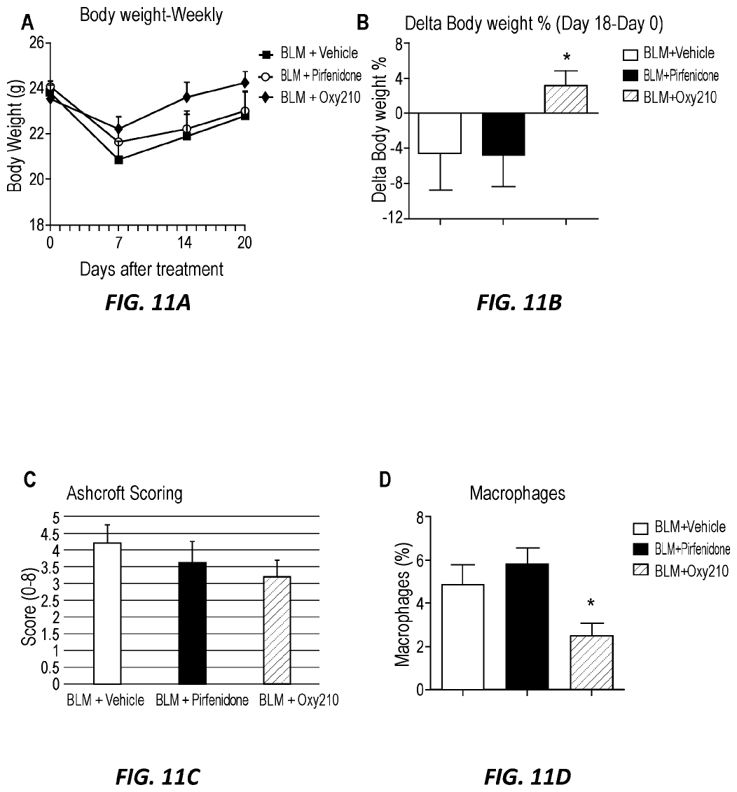

C57BL/6 mice were treated once intratracheally with bleomycin with or without pirfenidone or Oxy210. Body weights were measured during and at the end of the study (FIGS. 11A and 11B), Ashcroft scoring performed at the end of the study (FIG. 11C), and macrophage numbers in the bronchoalveolar lavage fluid (BALF) determined at the end of study (FIG. 11D).

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

1. A method for treating a fibrotic disease or disorder, comprising administering a therapeutically effective amount of a compound of Formula (I), or a pharmaceutically acceptable salt or solvate thereof, to a subject in need thereof, wherein the compound of Formula (I) has the structure: 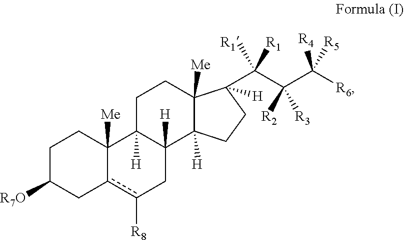
wherein: [symbol]
is a single or double bond;
R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl provided that one of R₁ and R₁′ is OH;
R₂, R₃, R₄, and R₈ are independently hydrogen, deuterium, C₁-C₈alkyl, or —OH, or one of R₂ or R₃ together with one of R₄ or R₅ forms a double bond;
R₆ is alkyl, aryl or heteroaryl, wherein the alkyl, aryl or the heteroaryl are optionally substituted with 1, 2, 3, or 4 R₉ groups;
R₇ is hydrogen, substituted or unsubstituted C₁-C₈alkyl, or —C(O)NR₁₀R₁₁;
R₈ is hydrogen or —OH;
each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄);
R₁₀ and R₁₁ are independently hydrogen, substituted or unsubstituted C₁-C₈alkyl, or substituted or unsubstituted aryl;
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

2. The method of claim 1, wherein the fibrotic disease or disorder is selected from the group consisting of acute interstitial pneumonitis, adhesive capsulitis, asthma, athrofibrosis (e.g., knee, shoulder, other joints), atrial fibrosis, bone-marrow fibrosis, cardiac fibrosis, chronic kidney disease, cirrhosis of liver and gallbladder, colloid and hypertrophic scar, Crohn's disease, cryptogenic organizing pneumonia, cystic fibrosis, desquamative interstitial pneumonia, diffuse parenchymal lung disease, Dupuytren's contracture, endomyocardial fibrosis, hypertrophic scarring, idiopathic interstitial fibrosis, idiopathic pulmonary fibrosis, interstitial lung disease, interstitial pneumonitis, lung fibrosis associated with chemotherapy or radiotherapy or antibiotic therapy, lung fibrosis associated with lung viral infection (for example coronavirus infection), ischemia associated fibrosis, keloid, liver fibrosis, non-alcoholic steatohepatitis (NASH), alcoholic steatohepatitis, liver fibrosis associated with chronic viral infection, lymphocytic interstitial pneumonia, mediastinal fibrosis, muscle fibrosis, myelofibrosis, nephrogenic systemic fibrosis, nonspecific interstitial pneumonia, organ transplant associated fibrosis, organ transplant fibrosis, pancreatic fibrosis, Peyronie's disease, progressive massive fibrosis, pulmonary fibrosis, renal fibrosis, respiratory bronchiolitis, retroperitoneal fibrosis, retroperitoneal fibrosis, scleroderma, systemic sclerosis, and vascular fibrosis.

3. The method of claim 1, wherein the fibrotic disease or disorder is pulmonary fibrosis, liver fibrosis, including non-alcoholic steatohepatitis (NASH), alcoholic steatohepatitis, idiopathic pulmonary fibrosis (IPF), kidney fibrosis, or cystic fibrosis.

4. The method of claim 1, wherein the fibrotic disease is due to injury or is idiopathic.

5. The method of claim 4, wherein the injury is an ischemic event or due to exposure to radiation, a chemical, or an infectious agent.

6. The method of claim 1, wherein the compound is administered after a fibrotic lesion has developed in the subject or prophylactically in patients with increased risk of developing fibrosis such as patients with metabolic diseases and obesity.

7. The method of claim 1, wherein the compound inhibits the expression of collagen, fibronectin, laminin, connective tissue growth factor (CTGF) and other extracellular matrix proteins associated with tissue fibrosis.

8. The method of claim 1, wherein said therapeutically effective amount is from about 1 μg/kg to 1000 mg/kg body weight of the subject administered once a day or multiple times a day.

9. The method of claim 1, wherein the compound is administered in a form selected from the group consisting of a solution, a suspension, a tablet, a pill, a capsule, and a sustained-release formulation.

10. The method of claim 1, wherein said administering is by injection, infusion, instillation, inhalation, or ingestion.

11. The method of claim 1, wherein said administering is intravenous, intramuscular, nasal, intratracheal, subcutaneous or subcuticular.

12. The method of claim 1, wherein said administering is systemic.

13. The method of claim 1, further comprising administering at least one additional therapeutic to the subject.

14. The method of claim 13 wherein the additional therapeutic is an anti-fibrotic agent.

15. The method of claim 13, wherein the additional therapeutic is selected the group consisting calcium channel blockers, cytotoxic agents, cytokines, chemokines, integrins, growth factors, hormones, lysophosphatidic acid (“LPA”) receptor 1 antagonists, agents that modulate the TGF-β pathway, endothelin receptor antagonists, agents that reduce connective tissue growth factor (“CTGF”) activity, matrix metalloproteinase (“MMP”) inhibitors, agents that reduce the activity of platelet-derived growth factor (“PDGF”), agents that interfere with integrin function, agents that interfere with the pro-fibrotic activities of cytokines, agents that reduce oxidative stress, PDE4 inhibitors, PDE5 inhibitors, mTor inhibitors, modifiers of the arachidonic acid pathway, peroxisome proliferator-activated receptor (“PPAR”)-γ agonists, kinase inhibitors, inhibitors of VEGF signaling pathway, matrix metalloproteinases, tissue inhibitors of metalloproteinases (“TIMPs”), HGF agonists, angiotensin-converting enzyme (“ACE”) inhibitors, angiotensin receptor antagonists, inhibitors of advanced glycation endproducts (“AGEs”) or their receptors (“RAGEs”), Rho kinase inhibitors, PKC inhibitors, ADAM-10 inhibitor, farnesoid X receptor agonists, caspase inhibitors, anti-oxidants, inhibitors of collagen expression, LMW heparin or heparin analogs, copper chelators, TNF-α blocking agents, agents that inhibit fibronectin deposition and/or enhance fibronectin degradation and turnover, HMG-CoA reductase inhibitors, Thy-1 (CD90) inhibitors, and antiviral agents.

16. The method of claim 1, wherein the subject is a mammal.

17. The method of claim 1, wherein the subject is human.

18. The method of claim 1, wherein the compound is Oxy210 having the structure [chemical structure]
